(19) United States
(12) Patent Application Publication
(10) Pub. No.: US 2025/0295818 A1
(43) Pub. Date: Sep. 25, 2025

(54) MICROFLUIDIC PREPARATION OF DUAL-PHASE NANODROPLETS WITH FLUORINATED COMPOUNDS

(71) Applicants: Bracco Suisse SA, Cadempino (CH); AVIGNON UNIVERSITÉ, Avignon CEDEX 1 (FR)

(72) Inventors: Romain MELICH, Choisy (FR); Philippe BUSSAT, Pers-Jussy (FR); Samir CHERKAOUI, Feigeres (FR); Christiane CONTINO-PEPIN, Althen des Paluds (FR); Stéphane DESGRANGES, Avignon (FR)

(21) Appl. No.: 18/864,464

(22) PCT Filed: May 11, 2023
(86) PCT No.: PCT/EP2023/062580

§ 371 Date: Nov. 8, 2024

(30) Foreign Application Priority Data
May 11, 2022 (EP) 22172772.0

(51) Int. Cl. A61K 49/22; A61K 9/51; A61K 41/00; B82Y 5/00

(52) U.S. Cl. CPC A61K 49/222; A61K 9/5123; A61K 9/5192; A61K 41/0028; A61K 2121/00; A61K 2123/00; B82Y 5/00

(57) ABSTRACT

The present invention relates to calibrated dual-phase nanodroplets comprising an outer layer and an inner core, said outer layer comprising a biocompatible fluorinated surfactant and said inner core comprising a fluorinated compound and a biocompatible oil. The invention further relates to a method of preparation of said calibrated dual-phase nanodroplets through microfluidic technique, and to their use for in vivo or in vitro diagnostic and/or for therapy.

### TECHNICAL FIELD

The invention generally relates to calibrated dual-phase nanodroplets stabilized by biocompatible fluorinated surfactants and having a core comprising a fluorinated compound and a biocompatible oil and their method of preparation through microfluidic technique. The invention further relates to the use of such calibrated nanodroplets for in vitro or in vivo diagnostic and/or for therapy.

### BACKGROUND OF THE INVENTION

Phase-change contrast agents (PCCAs) or acoustically activated nanodroplets are receiving increased popularity in both ultrasound diagnostic and therapeutic delivery. Except for the core, often consisting of liquid perfluorinated compounds, nanodroplets display similar composition to commercially available gas-filled microbubbles. Owing to Acoustic Droplet Vaporization (ADV) process, encapsulated droplets are converted into gas bubbles upon exposure to ultrasound energy beyond a vaporization threshold. In fact, ultrasounds act as a remote trigger to promote the vaporization of the droplets in a controllable, non-invasive and localized manner. Thanks to their smaller size compared to conventional microbubbles, nanodroplets display prolonged in vivo circulation and deep penetration into the tissues via the extravascular space. Moreover, below vaporization threshold, they are ultrasonically stable with low acoustic attenuation and can be acoustically vaporized at the location of interest.

Perfluorocarbon nanodroplets (“PFC-NDs”) present a real potential as an extravascular ultrasound contrast agent in numerous diagnostic and therapeutic applications including sonopermeabilization, blood brain barrier (BBB) opening, multimodal imaging modalities and to allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging (SRI) through localization since these agents can be activated and deactivated on demand by applying intermittent acoustic pulses.

In the last decade, the possibility to further add an oil phase in the composition of the nanodroplets, precisely into their inner core, has been investigated due to the advantage to combine the intrinsic properties of the perfluorocarbon-filled nanodroplets with those endowed by the presence of an oil in the core of such nanodroplets, such as encapsulating hydrophobic molecules for drug-delivery applications.

For instance, perfluorocarbon nanodroplets stabilized by the biocompatible fluorinated surfactant called “FTAC” and comprising an oil phase (i.e. triacetin) in their core have been reported by Astafyeva et al, 2015, who investigated perfluorocarbon emulsions as theranostic agents. In this work, ultrasonic homogenization was used to produce the perfluorocarbon nanoemulsions, followed by centrifugation and washing steps.

WO2016185425 teaches the synthesis and the use of DendriTAC as stabilizers in the preparation of perfluorocarbon nanoemulsions. Standard preparation methods, such as vortex, sonicator and microfluidizer (high pressure homogenizer) are proposed for the emulsion preparation. The possibility to use DendriTAC to stabilize nanodroplets additionally comprising an oil phase is also mentioned.

Al Rifai et al, 2020 discloses DendriTAC-stabilized nanodroplets also comprising an oil phase (i.e. tributyl O-acetylcitrate) in the perfluorocarbon core for the delivery of the hydrophobic drug Paclitaxel. For their preparation, standard emulsification technique combined with centrifugation procedures were used.

A major limitation of nanodroplets is their relatively limited physico-chemical stability over time, which may affect their use in diagnostic and therapy applications.

As reported in the literature, specific washing procedures are often needed at the end of the preparation of the dual-phase nanodroplets, in order to improve their sizes and sizes distribution profiles.

Both size and size distribution of nanodroplets are important quality attributes in determining the vaporization threshold, which corresponds to the value of ultrasound pressure required to convert a liquid core droplet into a gaseous bubble. In a polydisperse suspension, characterized by particles of varied sizes, nanodroplets with larger sizes, which require less energy to vaporize than smaller ones, influence the vaporization of the nanodroplets suspension.

On the contrary, in case of a monodisperse system containing particles of relatively uniform size, having a similar and uniform acoustic response to the ultrasound exposure, it is possible to apply the lowest acoustic pressure to achieve the highest vaporization efficiency.

More recently, microfluidics (MF) technology, also known as “lab on-a-chip”, has evolved as a powerful and scalable alternative for the consistent preparation of a large variety of size-controlled nanomedicines.

Melich et al, 2020 reports the use of rapid and controlled microfluidic mixing for the manufacturing of PFC-NDs.

Up to now, according to Applicants' knowledge, perfluorocarbon emulsions stabilized by biocompatible fluorinated surfactants and comprising a biocompatible oil have not been prepared yet through microfluidic techniques.

The Applicants have now developed a novel composition comprising calibrated dual-phase nanodroplets, stabilized by biocompatible fluorinated surfactants and having a core comprising a fluorinated compound and a biocompatible oil, said nanodroplets being obtained through microfluidic technique.

Generally, in the state of the art, the term “calibrated” is also indicated as “size-controlled”, “uniform-sized droplets”, “monodisperse(d)” or “monosize(d)”.

The Applicants observed that the presence of a biocompatible oil in the core of the calibrated nanodroplets may affect the properties of the calibrated nanodroplets manufactured according to the microfluidics techniques.

The inventors have in fact surprisingly found that improved stability properties of the calibrated dual-phase NDs can be obtained when adding a biocompatible oil in the core of the nanodroplets, as compared to conventional preparations method, generally based on multi-steps and tedious procedures.

### SUMMARY OF THE INVENTION

An aspect of the invention relates to a nanodroplet comprising an outer layer and an inner core, said outer layer comprising a biocompatible fluorinated surfactant and said inner core comprising a fluorinated compound and a triglyceride having a logP value higher than 5, wherein said biocompatible fluorinated surfactant is selected from:
(A) an amphiphilic dendrimer (Dendri-TAC);
(B) an amphiphilic linear oligomer (F-TAC)
or a mixture thereof.

In an embodiment, said fluorinated compound is a perfluorocarbon.

In a preferred embodiment, said triglyceride has a logP value higher than 7, still more preferably higher than 9, up to e.g. 25.

In an embodiment said triglyceride is selected from tricaprilin, trilaurin, triolein, trilinolein or a mixture thereof, preferred being tricaprilin.

A further aspect relates to an aqueous suspension comprising a plurality of nanodroplets as above defined, wherein said nanodroplets have a polydispersity index (PDI) lower than 0.25, preferably lower than 0.20, more preferably lower than 0.15, even more preferably lower than 0.10, and a Z-average diameter comprised between 100 nm and 1000 nm, preferably between 120 and 800 nm, more preferably between 150 and 400 nm.

A still further aspect relates to a method for the preparation of an aqueous suspension comprising a plurality of nanodroplets, said nanodroplets comprising an outer layer and an inner core, said outer layer comprising a biocompatible fluorinated surfactant as defined above, and said inner core comprising a fluorinated compound and a biocompatible oil having a logP value higher than 5, said method comprising the steps of:
a) preparing an aqueous phase;
b) preparing an organic phase,
wherein
i) said aqueous phase comprises a biocompatible fluorinated surfactant selected from Dendri-TAC, F-TAC or a mixture thereof and the organic phase comprises a fluorinated compound and a biocompatible oil; or
ii) said organic phase comprises a biocompatible fluorinated surfactant selected from Dendri-TAC, F-TAC or a mixture thereof, a fluorinated compound and a biocompatible oil;
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous; and
d) collecting said aqueous suspension from the exit channel of the microfluidic cartridge.

Preferably said biocompatible oil is a triglyceride.

More preferably said trygliceride has a logP value higher than 7, still more preferably higher than 9, up to e.g. 25.

Still more preferably, said triglyceride is selected from tricaprilin, trilaurin, triolein, trilinolein or a mixture thereof, preferred being tricaprilin.

According to a preferred embodiment, said aqueous phase comprises a biocompatible fluorinated surfactant selected from Dendri-TAC, F-TAC or a mixture thereof and said organic phase comprises a fluorinated compound and a biocompatible oil. Preferably, said aqueous suspension comprises a plurality of nanodroplets as above defined having a z-average diameter comprised between 100 nm and 1000 nm and a polydispersity lower than 0.25

Optionally after step d) the collected aqueous suspension is diluted.

A further aspect of the invention is related to a method for the preparation of an aqueous suspension suspension comprising a plurality of nanodroplets, said nanodroplets comprising an outer layer and an inner core, said outer layer comprising a biocompatible fluorinated surfactant and said inner core comprising a fluorinated compound and a biocompatible oil having a logP value higher than 5, said method comprising the steps of
a) preparing an aqueous phase comprising a biocompatible fluorinated surfactant;
b) preparing an organic phase comprising a fluorinated compound and a biocompatible oil having a logP value higher than 5;
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous suspension; and
d) collecting said aqueous suspension from the exit channel of the microfluidic cartridge.

A still further aspect relates to an aqueous suspension according to the invention for use in a diagnostic and/or therapeutic treatment.

### FIGURES

FIG. 1 is a schematic representation of the core portion of a microfluidic cartridge.

FIG. 2 shows a schematic representation of a cross-section of a staggered herringbone mixer (SHM) design.

### DETAILED DESCRIPTION OF THE INVENTION

The present invention relates to a novel composition comprising calibrated dual-phase nanodroplets stabilized by biocompatible fluorinated surfactants and having a core comprising a fluorinated compound and a biocompatible oil, preferably obtained through microfluidic technique. Said calibrated nanodroplets are suitable as contrast agents in ultrasound imaging techniques, known as Contrast-Enhanced Ultrasound (CEUS) Imaging, or in therapeutic applications, e.g. thermal ablation or for ultrasound mediated drug delivery.

### Dual-Phase Nanodroplets

In the present invention, the expression “dual-phase nanodroplets” refers to nanodroplets stabilized by biocompatible fluorinated surfactants, having a core comprising a fluorinated compound and a biocompatible oil, said nanodroplets being preferably obtained through microfluidic technique.

The term “calibrated” refers to the distribution of said dual-phase nanodroplets and indicates a polydispersity of a certain population of nanodroplets (e.g. with a z-average diameter comprised between 100 and 1000 nm) with a polydispersity index (PDI) lower than 0.25, preferably lower than 0.2, more preferably lower than 0.15, even more preferably lower than 0.1

The liquid core of the dual-phase nanodroplets is generally partitioned in two distinct phases: a first phase comprising the fluorinated compound(s) and a second phase comprising the oil(s), the two phases being substantially immiscible with each other.

This separation is related to the intrinsic properties of fluorinated compounds, being significantly more hydrophobic than hydrocarbons compounds, e.g. biocompatible oils, and lipophobic as well, repelling both water and lipids.

### Biocompatible Fluorinated Surfactants

As used herein, the term “biocompatible” indicates a compound and/or a composition having substantial compatibility with living tissue or a living system by not being toxic, injurious, or physiologically reactive and typically not causing immunological rejection.

In the present description and claims, the expression “surfactant” has its conventional meaning in the chemical field and refers to a compound suitable for forming the stabilizing layer of the nanodroplet.

The expression “fluorinated surfactant” refers to an amphiphilic organic compound suitable for forming the stabilizing layer of the nanodroplets, comprising a hydrophilic moiety and a hydrophobic moiety, said hydrophobic moiety comprising fluorine atoms (i.e. a fluorinated part).

The nanodroplets of the present invention are preferably dispersed in an aqueous solvent and stabilized by a layer which is composed of biocompatible fluorinated surfactants advantageously exhibiting a high affinity for both the inner core, comprising a fluorinated compound and a biocompatible oil, and the surrounding water.

In the present description and claims, the term Dendri-TAC refers to an amphiphilic dendrimer of generation n comprising:
a hydrophobic central core of valence 2 or 3;
generation chains attached to the central core and branching around the core; and
a hydrophilic terminal group at the end of each generation chain; wherein
n is an integer from 0 to 12;
the hydrophilic terminal group comprises:
a mono-, oligo- or polysaccharide residue,
a cyclodextrin residue,
a peptide residue,
a tris(hydroxymethyl)aminomethane (Tris), or
a 2-amino-2-methylpropane-1,3-diol;
the hydrophobic central core being a group of formula (Ia) or (Ib):

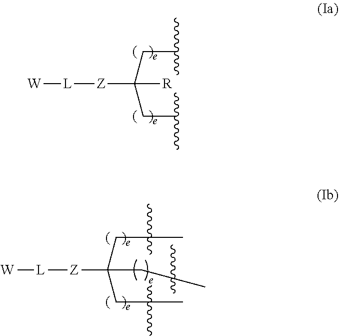

wherein:
W is R_{F} or a group selected from W₀, W₁, W₂ or W₃:

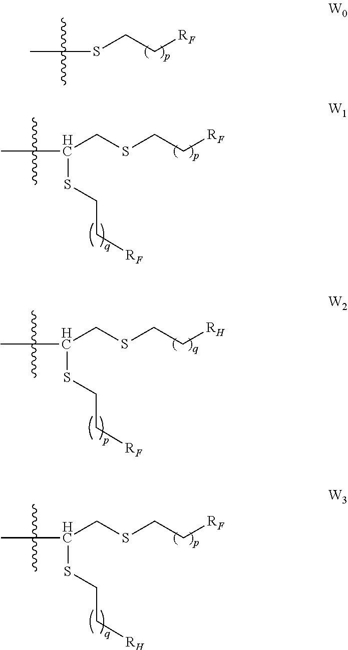

R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—,
Z is C(═O)NH or NHC(═O),
R is a C₁-C₆ alkyl group, and
e is at each occurrence independently selected from 0, 1, 2, 3 or 4.

In one embodiment, R_{F} is a C₄-C₁₀ perfluoroalkyl and R_{H} is a C₁-C₂₄ alkyl group. In this case, the hydrophobic central core of the amphiphilic dendrimer does comprise a perfluoroalkyl group, and said dendrimer is herein referred to as fluorinated amphiphilic dendrimer.

As used herein, the “valence m of the central core” refers to the number of generation chains attached to the central core, as illustrated in the following scheme 1:

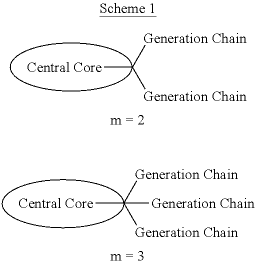

As used herein, a dendrimer of generation n=0, means that the m generation chains are connected to the central core through a first branching point (G₀), corresponding to the valence of the central core. A dendrimer of generation n=1 means that each of the m generation chains ramifies itself once, more specifically at the branching point G₁ (see scheme 2).

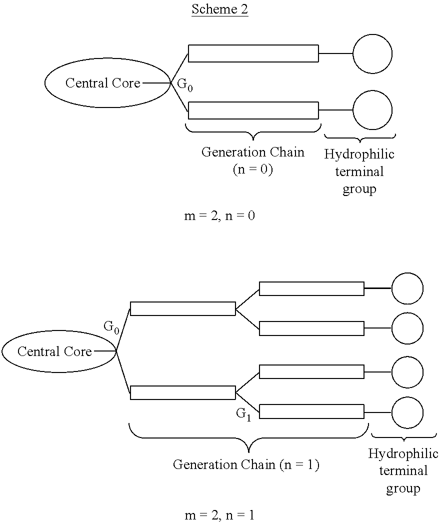

According to preferred embodiments, n is 0, 1 or 2, more preferably n is 0.

Each generation chain of the amphiphilic dendrimers according to the invention is ended by a hydrophilic terminal group.

In this respect, the mono-, oligo- or polysaccharide residue may be notably glucose, galactose, mannose, arabinose, ribose, maltose, lactose, hyaluronic acid.

The cyclodextrin residue may be selected from α, β or γ-Cyclodextrin.

The peptide residue may be chosen from linear or cyclic peptides containing the arginine-glycine-aspartic acid (RGD) sequence.

In another embodiment, there are included dendrimers wherein the generation chains are attached to the central core:
either via the group (a):

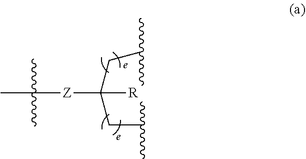

or via the group (b):

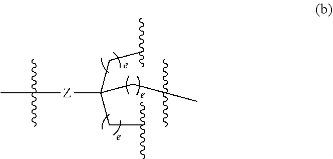

wherein
Z is C(═O)NH or NHC(═O), and is attached to the central core,
R is a C₁-C₆ alkyl group, and
e is at each occurrence independently selected from 0, 1, 2, 3 or 4.

In a further embodiment, there are included dendrimers wherein the central core is a group of formula (Ia) or (Ib):

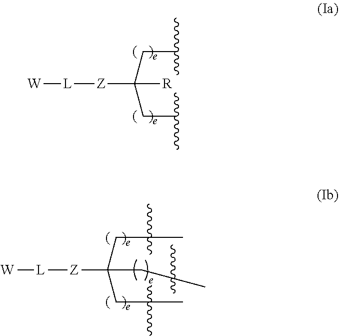

wherein:
W is R_{F} or a group selected from W₀, W₁, W₂ or W₃:

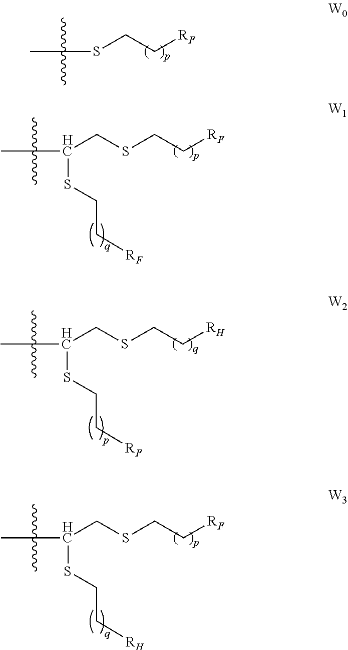

R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—.

In still a further embodiment, there are included dendrimers wherein WL is a group selected from:

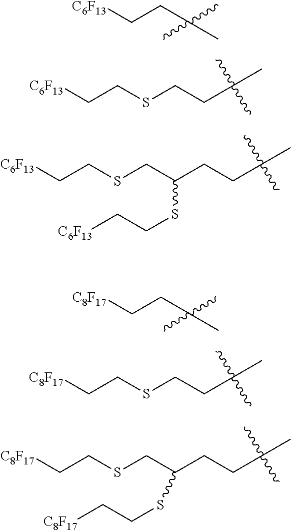

In yet another embodiment, there are included dendrimers wherein each generation chain (n) branches n times via a group (a) or a group (b) as defined above.

In another embodiment, there are included dendrimers wherein the terminal group comprises the following hydrophilic moieties:

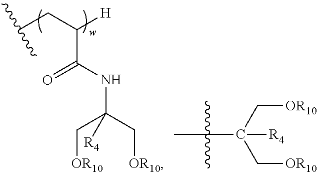

In a particular embodiment, there are included dendrimers having the following formula:

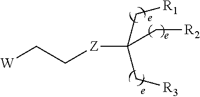

wherein:
W is R_{F} or a group selected from:

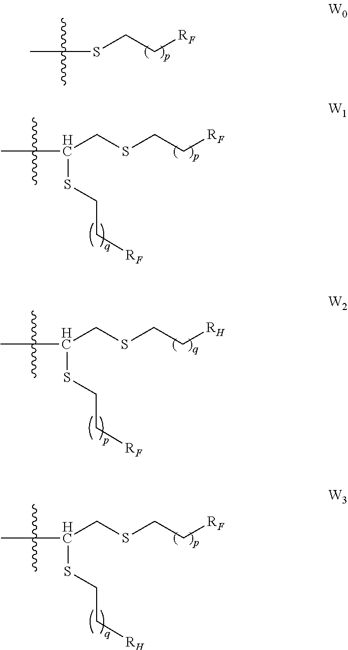

R_{F} being a C₄-C₁₀ perfluoroalkyl and R_{H} being a C₁-C₂₄ alkyl group,
p is 0, 1, 2, 3 or 4;
q is 0, 1, 2, 3 or 4;
Z is (CO)NH or NH(CO);
R₁, R₂, R₃ are H, or a group selected from (c) or (d):

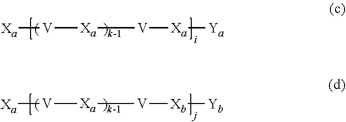

provided that:
R₁, R₂, R₃ are the same and selected from either group (c) or (d) or:
one of R₁, R₂, R₃ is H, the two others being the same and selected from either group (c) or (d);
X is Xₐ when j is 1 and X_{b} when j is 0;
Xₐ is at each occurrence independently selected from —OC(═O)CH₂—NH—, —OC(═O)CH₂—O—CH₂—, —O(CH₂)ᵣC(═O)—NH—, —O(CH₂)ᵣC(═O)—O—CH₂, OC(═O)NH—, —C(═O)—, —NH—, and —OCH₂—;
Yₐ is

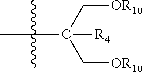

X_{b} is

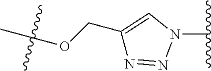 [SMILES: *OCc1cn(*)nn1]

Y_{b} is independently selected from:

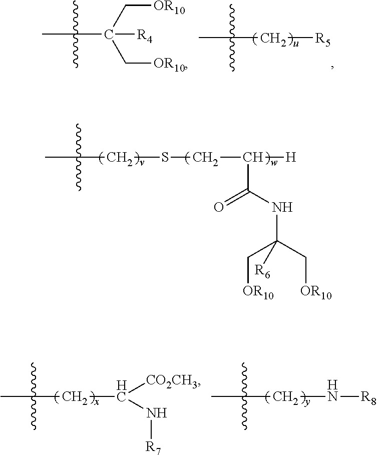

V is:

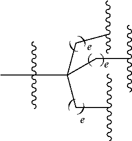

R₄, R₆ are each independently selected from H, C₁-C₆ alkyl or CH₂OR₁₀;
R₅ is a mono-, oligo-, polysaccharide or a cyclodextrin residue;
R₇, R₈ are each independently a peptide residue;
R₁₀ is H or a monosaccharide selected from glucose, galactose or mannose;
i is 0 or 1;
j is 0 or 1;
e is 0, 1, 2, 3 or 4;
k is an integer from 1 to 12, preferably from 1 to 5;
r is an integer from 1 to 10;
u is 0, 1, 2, 3 or 4;
v is 1, 2, or 3;
w is an integer from 1 to 20, preferably from 1 to 10;
x, y are each independently an integer from 1 to 6.

In a particular embodiment, there are included dendrimers having the following formula:

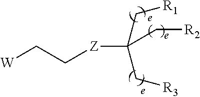

wherein:
   W is R_{F} or a group selected from:

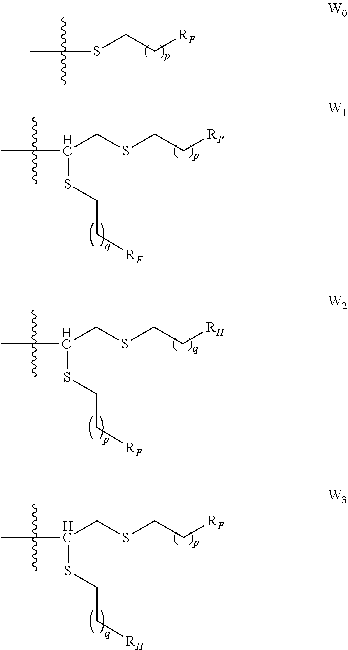

R_{F} being a C₁-C₂₄ perfluoroalkyl group, and R_{H} being a C₁-C₂₄ alkyl group,
p is 0, 1, 2, 3 or 4;
q is 0, 1, 2, 3 or 4;
Z is (CO)NH or NH(CO);
R₁, R₂, R₃ are H, or a group selected from (c) or (d):

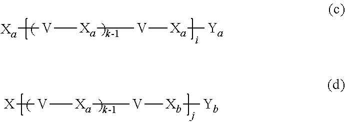

provided that:
R₁, R₂, R₃ are the same and selected from either group (c) or (d) or:
one of R₁, R₂, R₃ is H, the two others being the same and selected from either group (c) or (d);
X is Xₐ when j is 1 and X_{b} when j is 0;
Xₐ is at each occurrence independently selected from —OC(═O)CH₂—NH—, —OC(═O)CH₂—O—CH₂—, —O(CH₂)ᵣC(═O)—NH—, —O(CH₂)ᵣC(═O)—O—CH₂, OC(═O)NH—, —C(═O)—, —NH—, and —OCH₂—;
Yₐ is:

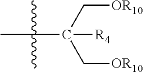

X_{b} is

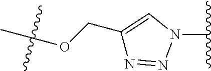 [SMILES: *OCc1cn(*)nn1]

Y_{b} is independently selected from:

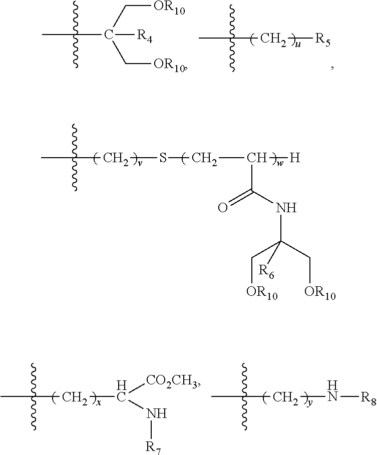

V is:

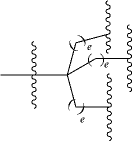

R₄, R₆ are each independently selected from H, C₁-C₆ alkyl or CH₂OR₁₀;
R₅ is a mono-, oligo-, polysaccharide or a cyclodextrin residue;
R₇, R₈ are each independently a peptide residue;
R₁₀ is H or a monosaccharide selected from glucose, galactose or mannose;
i is 0 or 1;
j is 0 or 1;
e is 0, 1, 2, 3 or 4;
k is an integer from 1 to 12, preferably from 1 to 5;
r is an integer from 1 to 10;
u is 0, 1, 2, 3 or 4;
v is 1, 2, or 3;
w is an integer from 1 to 20, preferably from 1 to 10;
x, y are each independently an integer from 1 to 6.

In a particular embodiment, the hydrophilic terminal group of the surfactants defined above is of following formula:

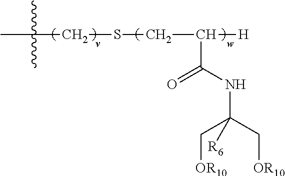

wherein R₆, R₁₀, v and w are as defined above, v being in particular equal to 3.

In a particular embodiment, the hydrophilic terminal group of the surfactants defined above is of following formula:

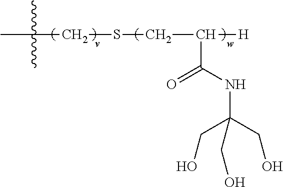

wherein v and w are as defined above, v being in particular equal to 3.

Suitable examples of amphiphilic dendrimers (Dendri-TAC) and preparation thereof are described in WO2016185425, and include F₆DiTAC₁₁, F₆DiTAC₆, F₆DiTAC₁₅, F₈DiTAC₅, DiF₆DiTAC₇, DiF₆DiTAC₁₅, DiF₈DiTAC₅, DiF₈DiTAC₁₁, with the following formulas:

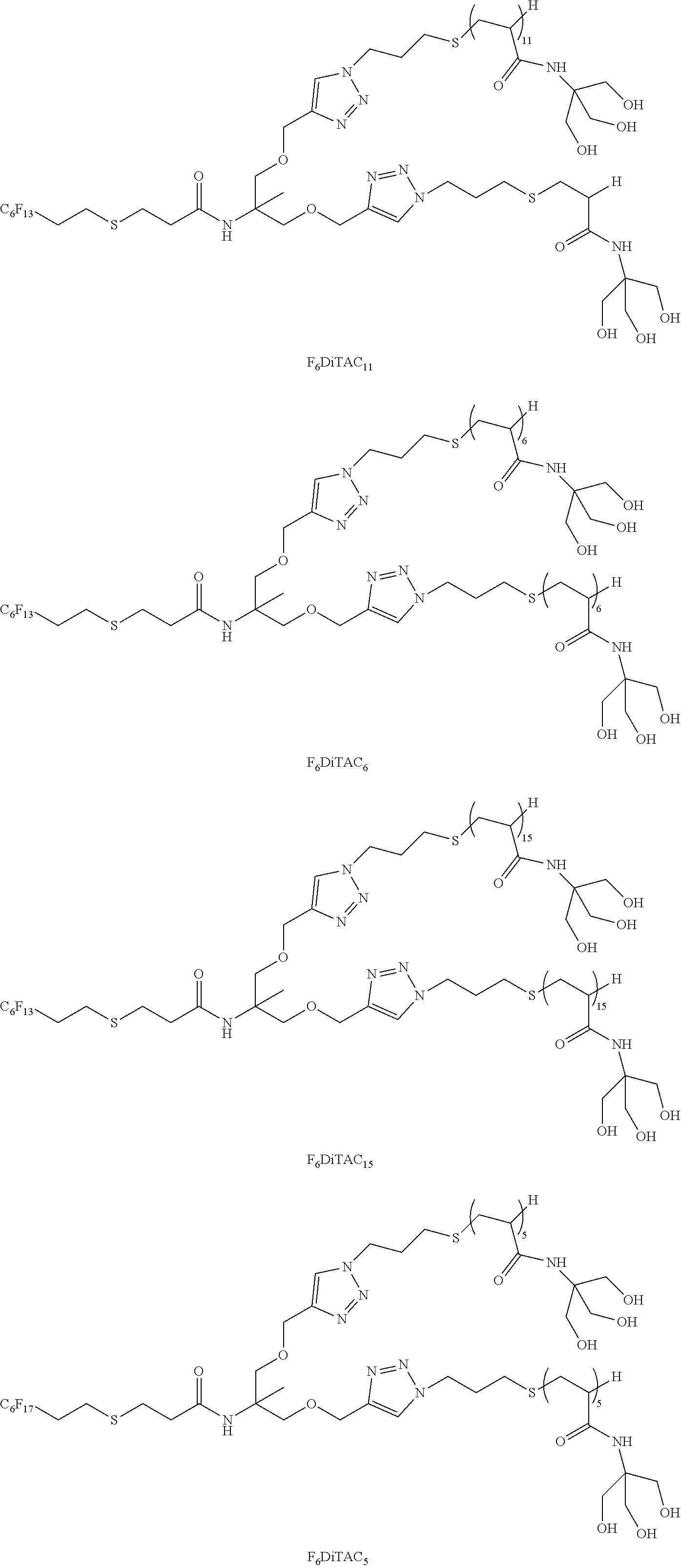

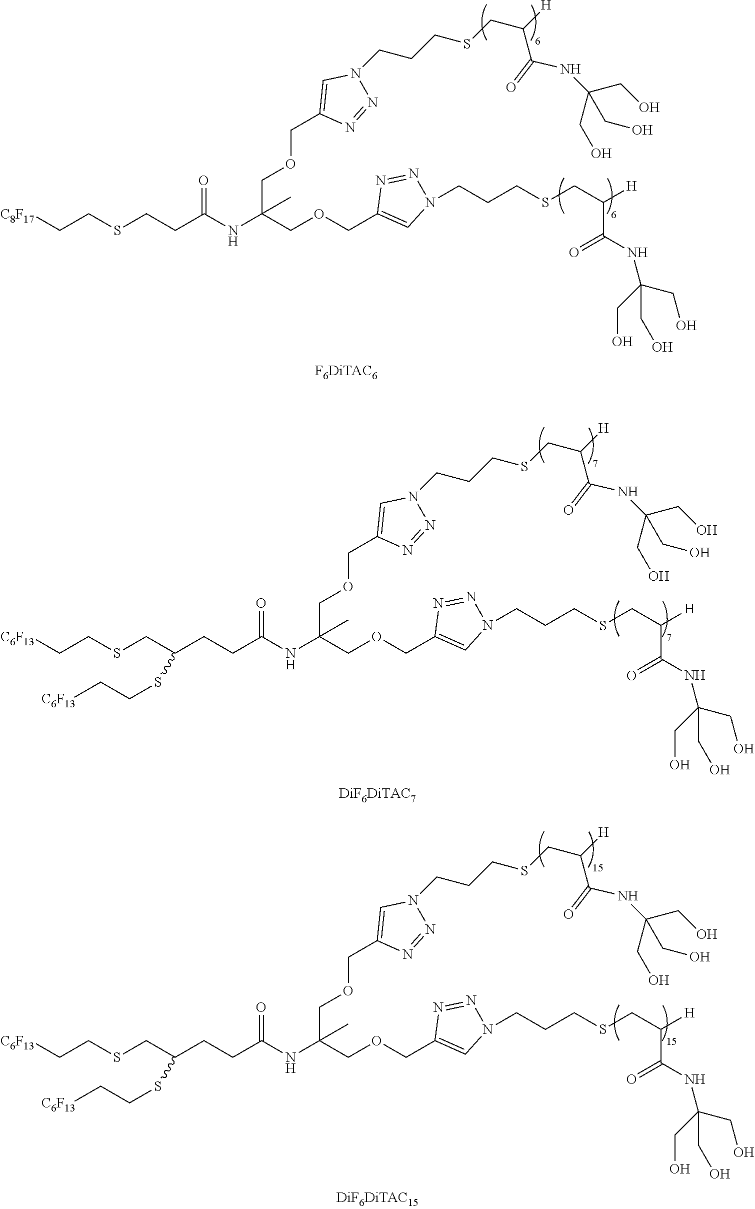

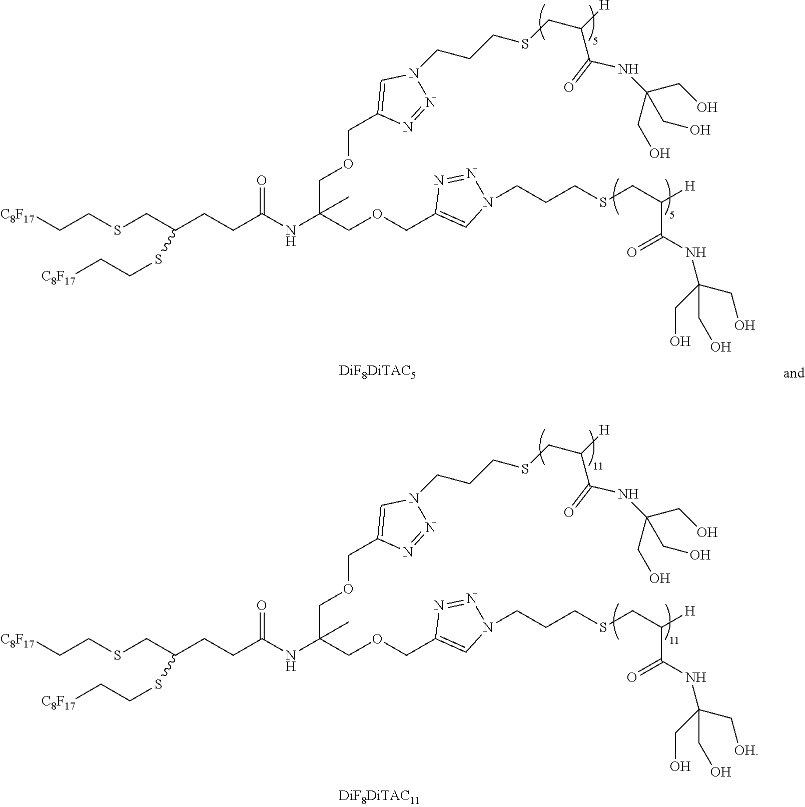

In a preferred embodiment of the present invention, the amphiphilic dendrimer Dendri-TAC is the following compound of formula IA

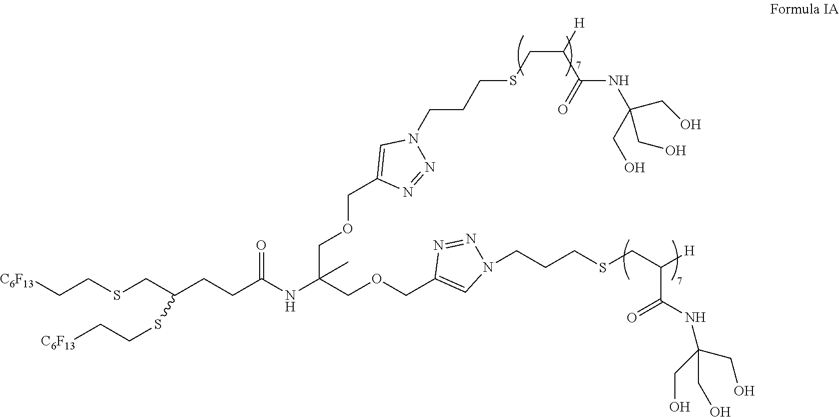

wherein the compound of formula IA is DiF₆DITAC₇.

As used herein, the term “F-TACs” comprise a hydrophilic part comprising an oligomer of polyTRIS type, and a hydrophobic part comprising a linear fluorinated alkyl chain.

In the present description and claims, the term F-TAC refers to a linear fluorinated surfactant having formula II:

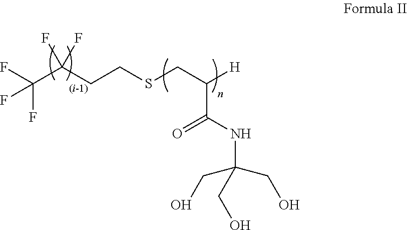

wherein:
n is the number of repeating Tris units (n=DPn is the average degree of polymerization), wherein the term Tris indicates the tris(hydroxymethyl)aminomethane unit, and
i is the number of carbon atoms in the fluoroalkyl chain.

In the present description and claims the compound of formula II can be interchangeably indicated as FᵢTACₙ, wherein:
n is the number of repeating Tris units (n=DPn is the average degree of polymerization) and
i is the number of carbon atoms in the fluoroalkyl chain.

According to an embodiment, i is comprised between 4 and 12, preferably between 6 and 10.

According to a further embodiment, when i is between 6 and 10, n is between 1 and 40, preferably between 4 and 30, still more preferably between 5 and 20.

According to a still further embodiment, when i is 8, n is between 1 and 40, for example between 4 and 30.

Suitable examples of amphiphilic linear oligomers F-TAC have been disclosed for instance in Astafyeva, 2015, and include F₈TAC₇, F₈TAC₁₈, F₈TAC₁₃ and F₆TAC₈ having the following formulas:

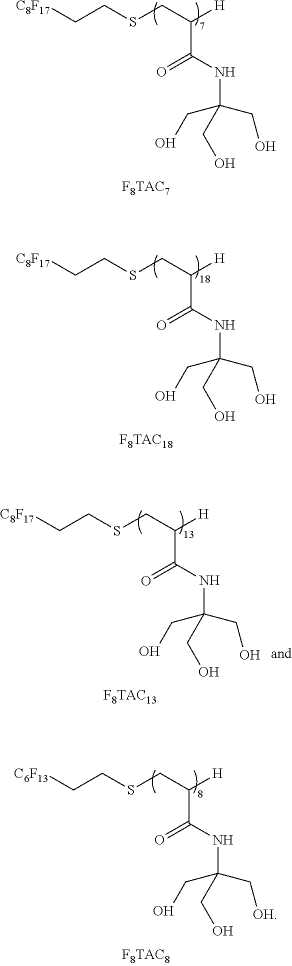

In an embodiment of the present invention, the amphiphilic linear oligomer (F-TAC) is the following compound of formula IIA

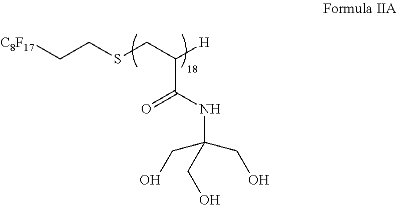

wherein the compound of formula IIA is F₈TAC₁₈.

The Applicants have now found that the physicochemical characteristics of the biocompatible fluorinated surfactants of the invention can influence the sizes of the disclosed (P) FC-NDs.

Table 1 shows selected physicochemical properties of preferred biocompatible fluorinated surfactants. In particular, the values of surface tension, critical micelle concentration and molecular weight have been reported.

TABLE 1
Physicochemical properties of biocompatible
fluorinated surfactants

| | ST | CMC | MW |
|---|---|---|---|
| Biocompatible fluorinated surfactants | (mN/m) | (mmol/L) | (g/mol) |
| F₈DiTAC₆ | 46.9 | 1.33E−02 | 2465 |
| DIF₆DiTAC₇ (Formula IA) | 41.9 | 2.42E−03 | 3471 |
| F₈TAC₇ | 32.3 | 1.34E−02 | 1705 |
| F₈TAC18 (Formula IIA) | 40.9 | 2.70E−02 | 3630 |

ST: surface tension at 25° C.;
CMC: critical micelle concentration;
MW: molecular weight

For instance, it has been observed that, in general, the lower the surface tension value associated to the biocompatible fluorinated surfactant, the smaller the NDs size.

In the present description and claims, the expression surface tension has its common meaning in the chemistry field and indicates the tendency of liquid surfaces to shrink into the minimum surface area possible. Surfactants, such as the disclosed biocompatible fluorinated surfactants, are compounds that lower the surface tension between two liquids, between a gas and a liquid, or between a liquid and a solid.

For instance, surface tension can be measured using the Wilhelmy plate technique, e.g. at the air/water interface, at (25.0±0.5)° C. with a K100 tensiometer (Kruss, Hamburg, Germany).

According to an embodiment, preferred biocompatible fluorinated surfactants are characterized by a surface tension value lower than 70 mN/m, more preferably lower than 50 mN/m.

### Fluorinated Compounds

In the present description and claims the term “fluorinated compounds” refers to a group of fluorine-containing compounds derived from (optionally substituted) hydrocarbons where hydrogen atoms have been partially or completely replaced by fluorine atoms, which are liquid at room temperature.

The expression “optionally substituted” refers to presence of functional groups, such as amines, ethers and halogen-containing groups.

Preferably the fluorinated compound is a perfluorocarbon (PFC), i.e. a fluorinated hydrocarbon where all the hydrogen atoms are substituted with fluorine atoms.

Liquid fluorinated compounds are characterized by a boiling point comprised between 25° C. and 160° C. In the present invention, the fluorinated compounds are preferably characterized by a boiling point comprised between 25° C. and 100° C., still more preferably between 27° C. and 60° C.

Suitable examples of fluorinated compounds are 1-Fluorobutane, 2-Fluorobutane, 2,2-Difluorobutane, 2,2,3,3-Tetrafluorobutane, 1,1,1,3,3-Pentafluorobutane, 1,1,1,4,4,4-Hexafluorobutane, 1,1,1,2,4,4,4-Heptafluorobutane, 1,1,2,2,3,3,4,4-Octafluorobutane, 1,1,1,2,2-Pentafluoropentane, 1,1,1,2,2,3,3,4-Octafluoropentane, 1,1,1,2,2,3,4,5,5,5-Decafluoropentane, 1,1,2,2,3,3,4,4,5,5,6,6-Dodecafluorohexane.

Suitable examples of perfluorocarbons are perfluoropentane, perfluorohexane, perfluoroheptane, perfluorooctane, perfluorononane, perfluorodecalin, perfluorooctylbromide (PFOB), perfluoro-15-crown-5-ether (PFCE), perfluorodichlorooctane (PFDCO), perfluorotributylamine (PFTBA), perfluorononane (PFN), and 1,1,1-tris(perfluorotert-butoxymethyl)ethane (TPFBME), or a mixture thereof.

In an embodiment said perfluorocarbon is preferably perfluoropentane (PFP) (boiling point 29° C.), perfluorohexane (PFH) (boiling point 57° C.) or perfluorooctylbromide (PFOB) (boiling point 142° C.).

### Oil Phase

In the present invention the calibrated nanodroplets are characterized by an inner liquid core comprising a fluorinated compound and an oil.

The oil can be a single oil or a mixture of different oils.

The term “oil” has its common meaning in the chemical field and refers to an organic compound, liquid at room temperature, selected among fatty acids, fatty acid esters or hydrocarbons. In the present invention said oil is a biocompatible oil (see above definition of biocompatible).

Examples of biocompatible oil include mono-, di- or tri-esters of glycerol, glycerol derivatives, mono-, di-, tri- or tetra-esters of citric acid, citric acid derivatives, fatty acids monoesters, sterol esters, sphingolipids, glycerophospholipids, saccharolipids, terpenes, lipid derivatives of prenol or a mixture thereof.

Said biocompatible oil can be a fatty acid ester, deriving from the combination of a fatty acid with an alcohol. Example of fatty acid esters are monoglycerides, diglycerides and triglycerides, which consist of a glycerol backbone with fatty acids, respectively one, two or three, esterified on the hydroxyl groups of the glycerol molecule.

Preferably said biocompatible oil is a triglyceride.

On the basis of the length of said fatty acids units, triglycerides can be classified as short-chain fatty acid with less than six carbons (e.g. triacetin and tributyrin), medium-chain fatty acid comprising from 6 to 12 carbons (e.g. tricaprilin), long-chain fatty acids comprising from 13 to 21 carbons (e.g. triolein) and very long-chain fatty acids from 22or more carbons (e. g. glyceryl tridocosahexaenoate).

Triglycerides can be also classified as saturated and unsaturated, wherein said unsaturated triglycerides tend to have a lower melting point than saturated analogues; as a result, unsaturated triglycerides are often liquid at room temperature.

In a preferred embodiment, said triglycerides are selected among triglycerides comprising medium-chain, unsaturated long-chain, unsaturated very long-chain fatty acids or a mixture thereof.

Preferably, said triglycerides comprise medium-chain fatty acids.

Still more preferably, said triglycerides are selected from tricaprilin, trilaurin, triolein, trilinolein or a mixture thereof, preferred being tricaprilin.

According to the present invention, other suitable examples of biocompatible oils can be trihexyl acetylcitrate, myristoleic acid, palmitoleic acid, oleic acid, gadoleic acid, erucic acid, ricinoleic acid, linoleic acid, eicosadienoic acid, alpha-linolenic acid, gamma-linolenic acid, arachidonic acid, coconut oil, soybean oil, castor oil, isopropyl palmitate, isopropyl myristate, squalene, squalane, vitamin E acetate and DL-alpha tocopherol.

The Applicants observed that the lipophilicity of the biocompatible oil, in particular of trygliceride, used in the preparation of the disclosed dual-phase nanodroplets plays a role in the stability of said nanodroplets.

One of the most applied ways to define the lipophilic nature of compounds is the partition coefficient, P, or its decimal logarithm, log P.

The “logP value” is a parameter indicating the partition coefficient (P) that describes the propensity of a neutral (uncharged) compound to dissolve in an immiscible biphasic system of lipid (e.g. fats, oils, organic solvents) and water. In other words, it measures how much of a solute dissolves in the water portion versus an organic portion. Solutes that are predominantly dissolved in the water layer are called hydrophilic and those predominantly dissolved in lipids are lipophilic.

The logP value is a constant defined in the following Equation 1:

$$Log P = log 10 ( [ organic ] / [ aqueous ] ) \qquad Eq . 1$$

wherein P indicates the partition coefficient and [ ] indicates the concentration of solute in the organic and aqueous partition.

A negative value for logP means the compound has a higher affinity for the aqueous phase (more hydrophilic); when logP=0 the compound is equally partitioned between the lipid and aqueous phases; a positive value for logP denotes a higher concentration in the lipidic phase (i.e. the compound is more lipophilic).

LogP values can be determined experimentally or computed. Different well-established methods are available for the experimental determination of LogP, such as shake-flask method and High-Performance Liquid Chromatography (HPLC).

The logarithm of the partition coefficient (logP) can be also computationally estimated by several methods.

In the present invention the logP values are based on the computation method XLOGP3, which predicts the logP value of a query compound by using the known logP value of a reference compound as a starting point. Additional details about the computation method XLOGP3 can be found in Cheng et al, 2007.

The Applicant observed that using a triglyceride characterized by a lower lipophilicity (e.g. logP lower than 5) led to a significantly reduced stability over time (e.g. higher % evol values).

On the other hand, triglycerides having higher lipophilicity (e.g. logP higher than 5) values endowed to more stable compositions of dual-phase nanodroplets.

In an embodiment, the biocompatible oil, in particular the triglyceride, has a logP value higher than 5, more preferably higher than 7, still more preferably higher than 9, up to 25.

In a further embodiment, said biocompatible oil is a mixture of oils.

In an embodiment said mixture comprises at least an oil having a logP value higher than 5, and at least an oil having a log P value lower than 5.

Preferably the amount of said oil having a log P higher than 5 is higher than 75% (mol), more preferably higher than 90%.

Preferably the amount of said oil having a log P lower than 5 is up to 25% (mol), more preferably up to 10%.

In certain embodiments the oil(s) having a logP higher than 5 represents the 100% (mol) of the biocompatible oil.

In an embodiment, said oil may additionally comprise a bioactive molecule (e.g. therapeutic or contrast agent) dispersed or dissolved therein. Bioactive molecule includes any molecule, compound, formulation or material capable of being used in the ultrasound-mediated therapeutic treatments in combination with dual-phase nanodroplets, which are capable of producing a biologically or therapeutically active effect on the region or organ to be treated.

Preferably, said bioactive molecule is a hydrophobic drug. Said bioactive molecule can be advantageously dissolved in the inner core of the dual-phase nanodroplets, in particular into the phase comprising the biocompatible oil(s).

### BFS/Fluorinated Compounds Molar Ratio

In the present description and claims the expression “BFS/fluorinated compoundmolar ratio” (Nr) indicate the ratio of biocompatible fluorinated surfactant and fluorinated compound that is used to prepare the disclosed dual-phase nanodroplets. It is possible to calculate the molar ratio by using the following formula:

$$Molar ratio Nr = \frac{total moles of biocompatible fluorinated surfactant}{total moles of fluorinated compound} \qquad Eq . 2 a$$

wherein:
the expression “total moles of biocompatible fluorinated surfactant” indicate the molar amount of biocompatible fluorinated surfactants used for the preparation of the nanodroplets suspension, and
the expression “total moles of fluorinated compound” indicates the molar amount of fluorinated compounds used for the preparation of the nanodroplets suspension.

Said molar amounts indicate the quantity of each component comprised in the respective aqueous and organic phases that are used for the preparation of the calibrated dual-phase nanodroplets.

In an embodiment the molar ratio between said biocompatible fluorinated surfactant and said fluorinated compound is comprised between 0.002 and 0.250, preferably between 0.005 and 0.100.

### Biocompatible Oil/Fluorinated Compound Molar Ratio

In the present description and claims the expression “Biocompatible oil/fluorinated compound molar ratio” (Nr) indicate the molar ratio of biocompatible oil and fluorinated compound that is used for the preparation of the disclosed dual-phase nanodroplets. It is possible to calculate the molar ratio by using the following formula:

$$Molar ratio Nr = \frac{total moles of biocompatible oil}{total moles of fluorinated compound} \qquad Eq . 2 b$$

wherein:
the expression “total moles of biocompatible oil” indicate the molar amount of biocompatible oil that is used for the preparation of the nanodroplets suspension, and
the expression “total moles of fluorinated compound” indicate the molar amount of (per) fluorinated compound forming the inner core of the NDs.

Said molar amounts indicate the quantity of each component comprised in the respective aqueous and organic phases that are used for the preparation of the calibrated dual-phase nanodroplets.

In an embodiment the molar ratio between said biocompatible oil and said fluorinated compound is comprised between 0.05 and 0.500, preferably between 0.07 and 0.400.

### Biocompatible Oil/Fluorinated Compounds Volume Ratio (v/v %)

In the present description and claims the expression “Biocompatible oil/fluorinated compound volume ratio” (v/v %) indicates the volume ratio of biocompatible oil and fluorinated compound that is used for the preparation of the disclosed dual-phase nanodroplets. It is possible to calculate the volume ratio by using the following formula:

$$Volume ratio ( \frac{v}{v} \% ) = \frac{( total volume of biocompatible oil )}{\begin{matrix}( total volume of biocompatible oil + \\ total volume of fluorinated compound )\end{matrix}} \times 100 \qquad Eq . 3$$

The volume ratio percentage indicates the volume of nanodroplet occupied by the biocompatible oil. For instance, a volume ratio of 50% means that 50% of the droplet core volume is occupied by the biocompatible oil.

The Applicant has observed that by using the disclosed microfluidic method is possible to obtain a dual-phase nanodroplet having a biocompatible oil/fluorinated compound volume ratio higher than 10%, preferably higher than 15%, more preferably higher than 20%, still more preferably higher than 30%, still more preferably higher than 40%, up to 50%.

### Calibrated Dual-Phase Nanodroplets

Another aspect relates to an aqueous suspension comprising a nanodroplet as above defined.

A further aspect relates to an aqueous suspension comprising a plurality of dual-phase nanodroplets as above defined, wherein said nanodroplets have a z-average diameter comprised between 100 nm and 1000 nm and a polydispersity lower than 0.25.

In the present description and claims, the term “plurality of dual-phase nanodroplets” refers to a population of nanodroplets characterized by a calibrated distribution, meaning that substantially all the nanodroplets have substantially similar sizes.

The expression “calibrated distribution” indicates a polydispersity of a certain population of nanodroplets (e.g. with a z-average diameter comprised between 100 and 1000 nm) with a polydispersity index (PDI) lower than 0.25, preferably lower than 0.2, more preferably lower than 0.15, even more preferably lower than 0.1.

The skilled person knows that the above numerical values are subject to experimental variability, which place limits on their accuracy. For this reason, in the present invention the error related to the experimental values is set to ±15%.

The term “polydispersity” (PDI) refers to a dimensionless measure of the broadness of the size distribution calculated from the cumulants analysis, wherein said cumulants analysis, defined in the International Standard on Dynamic Light Scattering ISO13321 (1996) and ISO22412 (2008), gives a mean particle size (z-average) and an estimate of the width of the distribution (polydispersity index).

For instance, a polydispersity higher than 0.7 indicates a very broad distribution of particles sizes, while a value lower than 0.08 indicates a nearly monodisperse sample characterized by a monomodal distribution. The polydispersity can be measured with the dynamic light scattering technique (DLS), by using for instance the Malvern Zetasizer Nano-ZS instrument (Malvern Instruments Ltd., UK).

The “z-Average Diameter (ZD)” is defined as the intensity-weighted mean diameter derived from the cumulants analysis. In other words, it relates to the average of calibrated nanodroplets size dispersed in the aqueous suspension measured through the dynamic light scattering technique (DLS).

In the present invention, the z-average diameter is comprised between 100 nm and 1000 nm, preferably between 120 and 800, more preferably between 150 and 400.

Suitable aqueous carriers for the aqueous suspension of the present invention, which are preferably physiologically acceptable, comprise water (preferably sterile water), aqueous solutions such as saline (which may advantageously be balanced so that the final product for injection is not hypotonic), or solutions of one or more tonicity adjusting substances. Tonicity adjusting substances comprise salts or sugars, sugar alcohols, glycols or other non-ionic polyol materials (e.g. glucose, sucrose, trehalose, sorbitol, mannitol, glycerol, polyethylene glycols, propylene glycols and the like), chitosan derivatives, such as carboxymethyl chitosan, trimethyl chitosan or gelifying compounds, such as carboxymethylcellulose, hydroxyethyl starch or dextran.

In the present invention, calibrated dual-phase nanodroplets are preferably produced by using a microfluidic technology.

### Stability

The Applicants have now surprisingly found that it is possible to substantially improve the stability of the disclosed calibrated dual-phase nanodroplets by adding an oil as defined above into the core of said nanodroplets.

In the present description and claims, the term “stability” indicates the property of a nanodroplets composition to substantially maintain over time its initial NDs sizes and preferably also its initial monodispersed distribution.

Initial NDs sizes and initial monodispersed distribution refer to the values of NDs sizes and monodispersity of the calibrated NDs composition at the end of the preparation process.

For the sake of clarity, the expression “end of the preparation process” refers either to i) the collection of the calibrated NDs from the exit channel of the microfluidic cartridge or ii) the collection of said calibrated NDs followed by a dilution step (i.e. step e).

Both these alternative final steps are performed before any storage period of the calibrated NDs.

The expression “storage period” indicates a period of time, e.g. expressed as hours, days or weeks, during which a microfluidically-prepared aqueous suspension of calibrated NDs is kept under certain conditions of temperature after the end of the preparation process.

It is possible to calculate the stability of the calibrated nanodroplets by using the NDs size evolution (% Evol) parameter, according to the following equation (Eq.3):

$$\% Evol = \frac{( Dfinal - Dinitial )}{Dinitial} \times 100 \qquad Eq . 4$$

wherein:
D final is the z-average diameter of the calibrated NDs after a certain time from the end of the preparation process, e.g. after 60 minutes from the end of preparation process or after a storage period (e.g. one week) at different conditions (different temperatures, pressure, etc.); and
D initial is the z-average diameter of the calibrated NDs immediately (e.g. within minutes) at the end of its preparation process.

In the present invention, a value of % Evol close to 0 (either positive or negative) indicates a higher stability of the calibrated NDs suspension, whereby the nanodroplets in the suspension substantially maintain over time their initial mean dimensions.

According to the present invention, the % Evol of a calibrated NDs suspension is preferably lower than ±50%, more preferably lower than ±30%, and still more preferably is lower than ±20%.

The Applicants have unexpectedly found that the presence of an oil into the core of said nanodroplets allows to reduce the % Evol and thus increases the stability of the calibrated NDs stabilized by BFS.

The Applicants have found that the % Evol parameter has a significant dependence from the presence of a biocompatible oil in the core composition of the nanodroplets. Improved stability may be achieved by using a biocompatible oil having a logP higher than 5, preferably higher than 7, even more preferably higher than 9, up to e.g. 25.

Moreover, it has been observed that using a suitable microfluidic preparation method is possible to obtain a ready-to-use suspension of calibrated dual-phase nanodroplets characterized by good values of PDI and improved stability over time, without the need to further submit the collected formulations to additional treatments, like centrifugations.

### Microfluidic Cartridge

The calibrated dual-phase NDs of the present invention are preferably produced through a bottom-up approach using a microfluidic technique.

In the present description and claims the expression “microfluidic technique” refers to a technology of manufacturing nanodroplets through a microfluidic cartridge designed to manipulate fluids in channels at the microscale.

Said microfluidic technique is a bottom-up approach, that is to say that the nanodroplets are obtained by assembling molecules (e.g. BFS, biocompatible oil and fluorinated compounds) into larger nanostructures (i.e. dual-phase nanodroplets).

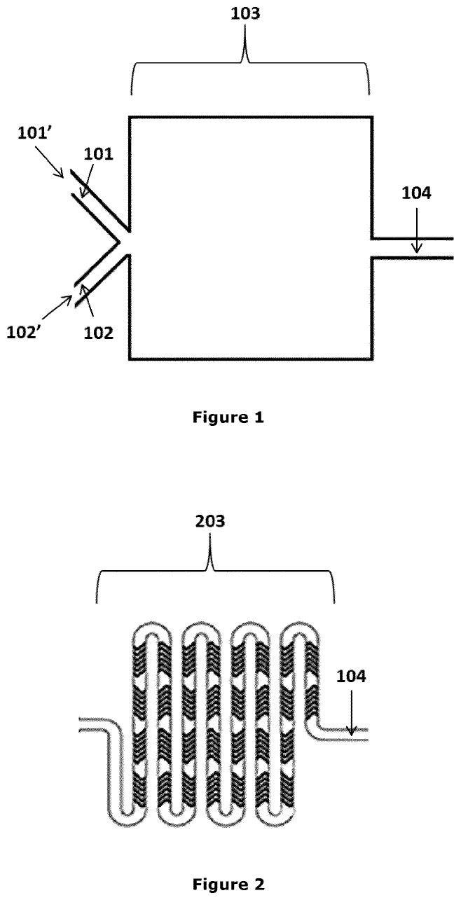

FIG. 1 shows a schematic representation of the core portion 100 of a microfluidic cartridge useful in the process of the invention. The cartridge comprises a first inlet 101 for feeding the aqueous phase 101′ and a second inlet 102 for supplying the organic phase 102′. The aqueous phase and the organic phase are directed towards a mixing device 103, for instance a staggered herringbone micromixer 203 as illustrated in FIG. 2, wherein they are mixed (e.g. through laminar mixing in the case of the micromixer of FIG. 2 endowing to the formation of NDs.

The calibrated fluorinated compound NDs are then directed to the exit channel 104, from where they are collected in a suitable container (e.g. a vial).

Alternatively, said microfluidic cartridge can be equipped with an additional channel, for instance placed between the mixing device 103 and the exit channel 104, aimed at diluting, with a suitable solvent, the calibrated fluorinated compound NDs suspension before their direction to the exit channel 104 (i.e. in-line dilution).

A mixing device 103 is generally characterized by suitable geometries able to enhance the microfluidic-mixing performance. In fact, the mixing process takes place into the peculiar micro-channel geometry of the mixing device, which causes fluid streams to mix together on the way to exit the microfluidic cartridge.

Different types of mixing devices are available with different shapes or microstructures. Suitable examples of mixing devices can be classified as passive micromixers, such as T and Y shaped mixers (e.g. staggered herringbone micromixer or toroidal mixer), and the mixer using flow focusing; and active micromixers, such as mixer using pressure field disturbance, electrokinetic active micromixer and ultrasound active micromixer.

Preferred in the present invention is a staggered herringbone micromixer (FIG. 2), wherein the mixing of the two liquid phases is controlled by lamination-mixing or a toroidal micromixer.

During the mixing phase the dual-phase nanodroplets are formed and directed to the exit channel of the microfluidic cartridge, or, alternatively, directed to an additional channel to dilute the nanodroplets before their direction to the exit channel.

In the present description and claims, the expression “exit (or outlet) channel” indicates the terminal portion of the microfluidic cartridge, toward which the just formed nanodroplets are directed from the mixing device and from where it is possible to collect the formed suspension of nanodroplets in a suitable container (e.g. a vial).

Typically, the operating pressure into the microfluidic cartridge is lower than 1000 psi (about 7000 kPa), preferably lower than 500 psi (about 3500 kPa), still more preferably lower than 300 psi (about 2000 kPa), still more preferably lower than 100 psi, (about 700 kPa), e.g. between 10 and 90 psi.

An example of microfluidic cartridge is the commercially available NxGen Cartridge, with or without in-line dilution, from Precision Nanosystems (Vancouver, Canada). These microfluidic cartridges can comprise either staggered herringbone or toroidal micromixers, both operating under non-turbulent conditions. For the manufacturing process, the microfluidic cartridge is mounted on a microfluidic instrument, generally equipped with a cartridge adapter, to host the microfluidic cartridge, and with containers (e.g. syringes or vials for continuous-flow injection) directly connected to the inlets of the microfluidic cartridge and specifically designed to pump the liquid phases into said inlets. Example of a microfluidic instrument is the NanoAssemblr® Benchtop Automated Instrument (Precision Nanosystems (Vancouver, Canada)).

An aspect of the invention relates to a method for the preparation of an aqueous suspension comprising a plurality of nanodroplets, said nanodroplets comprising an outer layer and an inner core, said outer layer comprising a biocompatible fluorinated surfactant as defined above, and said inner core comprising a fluorinated compound and a biocompatible oil having a logP value higher than 5, said method comprising the steps of:
a) preparing an aqueous phase;
b) preparing an organic phase,
   wherein
   i) said aqueous phase comprises a biocompatible fluorinated surfactant as defined above or a mixture thereof and the organic phase comprises a fluorinated compound and a biocompatible oil having a logP value higher than 5 or
   ii) said organic phase comprises a biocompatible fluorinated surfactant as defined above or a mixture thereof, a fluorinated compound and a biocompatible oil having a logP value higher than 5;
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous suspension and
d) collecting said aqueous suspension from the exit channel of the microfluidic cartridge.

According to a preferred embodiment, said aqueous phase comprises a biocompatible fluorinated surfactant selected from Dendri-TAC, F-TAC or a mixture thereof and said organic phase comprises a fluorinated compound and a biocompatible oil having a logP value higher than 5.

Preferably said fluorinated compound is a perfluorocarbon.

Preferably said biocompatible oil is a triglyceride having a logP value higher than 5.

Preferably said trygliceride has a logP value higher than 7, still more preferably higher than 9, up to e.g. 25.

Preferably, said triglyceride is selected from tricaprilin, trilaurin, triolein, trilinolein or a mixture thereof, preferred being tricaprilin.

Preferably, said aqueous suspension comprises a plurality of nanodroplets as above defined, wherein said nanodroplets have a z-average diameter comprised between 100 nm and 1000 nm and a polydispersity lower than 0.25, preferably lower than 0.20, more preferably lower than 0.15, even more preferably lower than 0.1

In a further embodiment, said biocompatible oil is a mixture of oils.

In an embodiment said mixture comprises at least an oil having a logP value higher than 5, and at least an oil having a log P value lower than 5.

Preferably the amount of said oil having a log P higher than 5 is higher than 75% (mol), more preferably higher than 90%.

Preferably the amount of said oil having a log P lower than 5 is up to 25% (mol), more preferably up to 10%.

Typically, at step c) the injection of the aqueous phase and the injection of the organic phase are carried out simultaneously.

The expression “simultaneously” indicates the simultaneous injection (i.e. co-injection) of the aqueous phase and the organic phase into the microfluidic cartridge, that is to say that the aqueous phase and organic phase are injected into two separate inlets of the microfluidic cartridge at the same time or at substantially the same time (e.g. within few seconds).

According to the disclosed method it is possible to obtain an aqueous suspension of calibrated dual-phase nanodroplets by a single passage of the liquid phases through the two-channel microfluidic system.

In an embodiment said method for the preparation of an aqueous suspension of calibrated dual-phase nanodroplets is the microfluidic technique, wherein said calibrated nanodroplets (Z-average diameter comprised between 100 and 1000 nm) have a polydispersity index (PDI) lower than 0.25, preferably lower than 0.20, more preferably lower than 0.15, even more preferably lower than 0.1.

Advantageously, when the surfactant is dissolved in the aqueous phase, the present novel method can be used for the preparation of an aqueous-suspension of calibrated nanodroplets stabilized by any other biocompatible surfactants, particularly biocompatible fluorinated surfactants, other than Dendri-TAC and FTAC.

As mentioned above, the expression “biocompatible fluorinated surfactant” refers to amphiphilic organic compounds suitable for forming the stabilizing layer of a nanodroplet, comprising a hydrophilic moiety and a hydrophobic moiety. Said amphiphilic organic compounds have substantial compatibility with living tissue or a living system by not being toxic, injurious, or physiologically reactive and typically not causing immunological rejection.

A further aspect of the invention is related to a method for the preparation of an aqueous suspension suspension comprising a plurality of nanodroplets, said nanodroplets comprising an outer layer and an inner core, said outer layer comprising a biocompatible fluorinated surfactant and said inner core comprising a fluorinated compound and a biocompatible oil having a logP value higher than 5, said method comprising the steps of
a) preparing an aqueous phase comprising a biocompatible fluorinated surfactant;
b) preparing an organic phase comprising a fluorinated compound and a biocompatible oil having a logP value higher than 5;
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous suspension; and
d) collecting said aqueous suspension from the exit channel of the microfluidic cartridge.

Preferably, said aqueous suspension comprises a plurality of nanodroplets as above defined, wherein said nanodroplets have a z-average diameter comprised between 100 nm and 1000 nm and a polydispersity lower than 0.25, preferably lower than 0.20, more preferably lower than 0.15, even more preferably lower than 0.1

### Aqueous Phase

The expression “aqueous phase” refers to a liquid comprising an aqueous liquid component, including for instance, water, aqueous buffered solutions, aqueous isotonic solutions or a mixture thereof. Preferably the aqueous phase is water.

According to a preferred embodiment, said aqueous phase comprises a biocompatible fluorinated surfactant selected from the amphiphilic linear oligomers F-TAC and the amphiphilic dendrimers Dendri-TAC, as described above, or a mixture thereof.

For instance, a biocompatible fluorinated surfactant can be admixed with an aqueous component through traditional techniques (e.g. stirring) in order to prepare the aqueous phase to be injected into the first inlet of the microfluidic cartridge.

At step a) the aqueous phase comprises a biocompatible fluorinated surfactant at a concentration ranging between 0.0006 mmol/mL and 0.006 mmol/mL, more preferably between 0.0001 mmol/mL to 0.015 mmol/mL, still more preferably between 0.001 mmol/mL and 0.01 mmol/mL.

### Organic Phase

The expression “organic phase” refers to a liquid comprising an organic solvent miscible with water including methanol, ethanol, isopropanol, acetonitrile and acetone. Preferably the organic phase is ethanol.

According to a preferred embodiment, said organic phase comprises:
a fluorinated compound or a mixture of different fluorinated compounds, and
a biocompatible oil having a logP higher than 5 or a mixture of different biocompatible oils,
   wherein said biocompatible oil is substantially not soluble in said fluorinated compound.

Due to its solubility profile, the biocompatible oil forms a separated phase in the inner core of the nanodroplet, said inner core being thus partitioned in two distinct phases: a liquid phase comprising the fluorinated compound and an oil phase.

Preferably the fluorinated compounds are perfluorocarbons.

Preferably said biocompatible oil has a logP value higher than 5, more preferably higher than 7, still more preferably higher than 9, up to e.g. 25. Still more preferably said biocompatible oil is a triglyceride.

Suitable examples of fluorinated compounds and biocompatible oils are those mentioned above.

As an example, a fluorinated compound and a biocompatible oil can be admixed with an organic solvent through traditional techniques (e.g. stirring) in order to prepare the organic phase to be injected into the second inlet of the microfluidic cartridge.

In an embodiment, at step b) the organic phase comprises a fluorinated compound at a concentration ranging between 0.003 mmol/mL and 0.142 mmol/mL, more preferably between 0.011 mmol/mL and 0.085 mmol/mL, still more preferably between 0.013 mmol/mL to 0.057 mmol/mL.

In a further embodiment, at step b) the organic phase comprises a biocompatible oil at a concentration ranging between 0.1 μmole/mL and 40 μmole/mL, more preferably between 0.5 μmole/mL and 25 μmole/mL, still more preferably between 1 μmole/mL and 15 μmole/mL.

In an alternative embodiment, said organic phase comprises:
a biocompatible fluorinated surfactant as defined above,
a fluorinated compound, and
a biocompatible oil.

For instance, said organic phase comprises
i) a biocompatible fluorinated surfactant at a concentration ranging between 0.0006 mmol/mL and 0.006 mmol/mL, more preferably between 0.0001 mmol/mL to 0.015 mmol/mL, still more preferably between 0.001 mmol/mL and 0.01 mmol/mL;
ii) a fluorinated compound at a concentration ranging between 0.003 mmol/mL to 0.142 mmol/mL, more preferably between 0.011 mmol/ml and 0.085 mmol/mL, still more preferably between 0.013 mmol/mL to 0.057 mmol/mL; and
iii) a biocompatible oil at a concentration ranging between 0.1 μmole/mL and 40 μmole/mL, more preferably between 0.5 μmole/mL and 25 μmole/mL, still more preferably between 1 μmole/mL and 15 μmole/mL.

In a still further embodiment of the invention both the aqueous and organic phases are preferably injected at a temperature lower than room temperature (e.g. from about 4° C. to 20° C.) into the microfluidic cartridge, to avoid vaporization of fluorinated compounds having a boiling point close to room temperature.

A further aspect relates to an aqueous suspension comprising a plurality of nanodroplets, said nanodroplets comprising an outer layer and an inner core, said outer layer comprising a biocompatible fluorinated surfactant as above defined, and said inner core comprising a fluorinated compound and a biocompatible oil having a logP value higher than 5, obtainable by a method of preparation comprising the steps of:
a) preparing an aqueous phase;
b) preparing an organic phase,
wherein i) said aqueous phase comprises a biocompatible fluorinated surfactant, selected from Dendri-TAC, F-TAC or a mixture thereof, and the organic phase comprises a fluorinated compound and a triglyceride having a logP value higher than 5, or
ii) said organic phase comprises a biocompatible fluorinated surfactant, selected from selected from Dendri-TAC, F-TAC or a mixture thereof, a fluorinated compound and a triglyceride having a logP value higher than 5.
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous suspension; and
d) collecting said aqueous suspension from the exit channel of the microfluidic cartridge; and

Preferably said triglyceride has logP value higher than 7, still more preferably higher than 9, up to e.g. 25.

In a preferred embodiment, said trygliceride is selected among from tricaprilin, trilaurin, triolein, trilinolein or a mixture thereof, preferred being tricaprilin.

In a preferred embodiment, said aqueous phase comprises a biocompatible fluorinated surfactant selected from the selected from Dendri-TAC, F-TAC or a mixture thereof, and the organic phase comprises a fluorinated compound, being preferably a perfluorocarbon, and a trygliceride having a logP value higher than 5.

In an embodiment, said organic phase further comprises a hydrophobic drug. Preferably, said aqueous suspension comprises a plurality of nanodroplets as above defined, wherein said nanodroplets have a z-average diameter comprised between 100 nm and 1000 nm and a polydispersity lower than 0.25, preferably lower than 0.20, more preferably lower than 0.15, even more preferably lower than 0.1.

### Total Flow Rate (TFR) and Flow Rate Ratio (FRR)

The method of the present invention allows to control the dual-phase nanodroplets characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 4:

$$Flow rate ratio = \frac{volume of aqueous phase}{volume of organic phase} \qquad Eq . 5$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1, more preferably the FRR is 1:1.

In the present invention, the respective concentrations of biocompatible fluorinated surfactant, fluorinated compound and biocompatible oil can be purposely tuned in order to obtain specific molar ratio between said biocompatible fluorinated surfactant and said fluorinated compound and between said biocompatible oil and said fluorinated compound

Advantageously at the end of the disclosed process it is possible to successfully obtain a ready-to-use composition of calibrated dual-phase nanodroplets characterized by an improved stability overtime.

The Applicants have demonstrated that by a single passage into the microfluidic cartridge, it is possible to produce stable calibrated dual-phase nanodroplets without the need of additional purification processes, such as multiple centrifugation treatments, as reported in prior art.

Calibrated dual-phase nanodroplets obtained by the disclosed microfluidic method present advantages over similar formulations obtained by conventional preparation processes, such as increased monodispersity and improved stability. Moreover, the disclosed microfluidic method endows to directly collect from the microfluidic cartridge said monodisperse and stable calibrated dual-phase nanodroplets without the need to perform any purification steps, such as centrifugation procedure.

### Optional Step e) Dilution

According to present invention, the method of preparation further comprises an optional step e), which comprises diluting the collected aqueous suspension of calibrated dual-phase nanodroplets.

In the present description and claims the term “dilution” refers to the process of reducing the concentration of calibrated nanodroplets in the suspension, by adding a suitable amount of aqueous liquid, including water or an aqueous solution.

A suitable amount of aqueous liquid corresponds to the quantity of aqueous liquid necessary to reduce the concentration of the calibrated nanodroplets in the aqueous suspension from 2 to 10-folds.

In a preferred embodiment, the optional step e) of the present method comprises diluting the collected aqueous suspension of calibrated nanodroplets from 1 to 20-folds, preferably from 3 to 8-folds, still more preferably the collected aqueous suspension is diluted 5-fold.

An additional effect of dilution is that of reducing the relative amount of organic solvent in the suspension.

In a further preferred embodiment, the step e) of the present method comprises diluting the collected suspension of calibrated nanodroplets with water.

As mentioned above, the diluting step can be alternatively performed inside the microfluidic cartridge, due to an additional channel (e.g. placed between the mixing device 103 and the exit channel 104 in FIG. 1) aimed at diluting the calibrated NDs suspension before their direction to the exit channel.

In this case, the step e) of the present method comprises diluting the suspension of calibrated nanodroplets from 1 to 20-folds, preferably from 3 to 8-folds, still more preferably the collected aqueous suspension is diluted 5-fold, before their collection from the microfluidic cartridge.

### Acoustic Droplet Vaporization

The expression “acoustic droplet vaporization (ADV)” refers to the phase-shift of the inner core of a nanodroplet comprising a fluorinated compound as above defined from liquid to gas state as a result of an applied ultrasound energy beyond a vaporization threshold.

Said ultrasound act as an external stimulus to promote the vaporization of the droplets in a controllable, non-invasive and localized manner.

Below vaporization threshold, the nanodroplets are ultrasonically stable with low acoustic attenuation and can be acoustically vaporized at the location of interest. Thanks to their smaller size and volume compared to conventional microbubbles, nanodroplets display prolonged in vivo circulation, deep penetration into the tissues via the extravascular space (Helfield et al. 2020).

### Use

Calibrated dual-phase nanodroplets can represent an alternative to gaseous microbubbles for medical (therapeutic) ultrasound applications. Upon applying ultrasound energy, droplets can be selectively vaporized in a region of interest to form microbubbles. After activation, the calibrated dual-phase nanodroplets can substantially be used in the same manner as conventional Contrast-Enhanced UltraSound (CEUS).

An aspect relates to an aqueous suspension obtained according to the process as above defined for use in a diagnostic and/or therapeutic treatment.

Diagnostic exam includes any method where the use of the dual-phase nanodroplets allows enhancing the visualisation of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation).

Therapeutic treatment includes any method of treatment of a patient. In preferred embodiments, the treatment comprises the combined use of ultrasounds and dual-phase nanodroplets either as such (e.g. in ultrasound mediated thrombolysis, high intensity focused ultrasound ablation, blood-brain barrier permeabilization, immunomodulation, neuromodulation, radiosensitization) or in combination with a therapeutic agent (i.e. ultrasound mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the nanodroplets may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound mediated delivery).

The following examples will help to further illustrate the invention.

### EXAMPLES

### Materials and Methods

The following materials are employed in the subsequent examples:

| | |
|---|---|
| DiF₆DiTAC₇ | Formula IA |
| F₈TAC₁₈ | Formula IIA |
| Perfluoropentane | 288 g/mol, bp: 28-30° C. - Interchim |
| (Dodecafluoropentane) | (Montluçon, France) |
| Ethanol | 96% Thommen-Furler AG |
| Tricaprilin | C₂₇H₅₀O₆, 470 g/mol, 99% - Sigma |
| Tributyrin | C₁₅H₂₆O₆, 302 g/mol, 97% - Fluka |
| DIO | 882 g/mol - Molecular Probes, Invitrogen |

### Example 1

### Preparation of Dual-Phase Nanodroplets Using a Microfluidic Platform

Dual-phase nanodroplets were formulated with a NanoAssemblr™ Benchtop automated instrument from Precision Nanosystems (Vancouver, Canada) equipped with a staggered herringbone micromixer (SHM) allowing size-controlled self-assemblies. Briefly, an aqueous phase comprising a biocompatible fluorinated surfactant (BFS) was injected into the first inlet whereas the organic phase composed of a perfluorocarbon (PFC) and a biocompatible oil, both dissolved in ethanol, was injected into the second inlet of the microfluidic cartridge (FIG. 1). Both phases were placed into an ice bath at about 4° C. before the NDs formulation. Microscopic characteristics of the channels are engineered to cause an accelerated mixing of the two fluid streams in a controlled fashion. The microfluidic process settings, namely the Total Flow Rate (TFR, in mL/min) and the Flow Rate Ratio (FRR), were varied to control the NDs characteristics. The NDs suspensions were collected from the exit channel in a Falcon vial (15 mL) and 5-fold diluted with ultrapure water.

### Example 2

### Effects of the Presence of a Biocompatible Oil in the Inner Core of the Calibrated Nanodroplets

The effects of the presence of a biocompatible oil in the inner core of the microfluidically-obtained nanodroplets were investigated.

For this purpose, several compositions comprising a nanodroplets suspension stabilized by the Dendri-TAC surfactant DiF₆DiTAC₇ and having a core comprising perfluoropentane as PFC were prepared and characterized, with and without the addition of tricaprilin as biocompatible oil.

Said compositions were prepared as described in Example 1, and the processing parameters were set as to obtain different molar ratios (from low to high) between BFS and PFC for each composition. For this purpose, three different FRR were tested: 3:1. 2:1 and 1:1. The TFR was 10 mL/min. The concentration of the BFS in the aqueous phase was 1 mg/ml and that of perfluoropentane in the organic phase was 10 μL/mL.

For the formulations comprising the biocompatible oil, three different concentrations of tricaprilin in organic phase were tested, namely 2 μL/mL, 5 μL/mL and 10 μL/mL and were compared to a formulation without oil. As described in Example 1, in order to obtain the calibrated dual-phase nanodroplets, the biocompatible oil was added in the organic phase together with the selected perfluorocarbon, before the injection into the microfluidic cartridge.

A summary of all the Compositions obtained is listed in Table 2, wherein the quantities of biocompatible oil, the BFS/PFC molar ratios and the respective FRRs are displayed in the first left columns.

The obtained formulations were characterized using a Malvern Zetasizer Nano-ZS instrument (Malvern Instruments Ltd., UK), measuring sizes (Z-average) and polydispersity (PDI) over time after a storage of one week at 4° C.

Each measurement was performed at room temperature (i.e. 25° C.).

### Results

TABLE 2a
Effects of the presence of a biocompatible oil
in the inner core of calibrated nanodroplets

| | | Oil | | Molar | | | % |
|---|---|---|---|---|---|---|---|
| | | (μL/ | | ratio Nr | Size | | evol |
| | | mL) | FRR | (BFS/PFC) | (nm) | PDI | (%) |
| Composition 1 | A | NO | 1:1 | 0.025 | 270 | 0.07 | 99 |
| | B | 2 | | | 897 | 0.179 | 2 |
| | C | 5 | | | 680 | 0.258 | −17 |
| | D | 10 | | | 303 | 0.16 | −6 |
| Composition 2 | A | NO | 2:1 | 0.051 | 198 | 0.108 | −85 |
| | B | 2 | | | 178 | 0.186 | −1 |
| | C | 5 | | | 150 | 0.218 | 2 |
| | D | 10 | | | 156 | 0.227 | 7 |
| Composition 3 | A | NO | 3:1 | 0.076 | 194 | 0.098 | 52 |
| | B | 2 | | | 177 | 0.191 | −3 |
| | C | 5 | | | 144 | 0.257 | 1 |
| | D | 10 | | | 139 | 0.23 | 11 |

The overall results of the study of the effects of the addition of a biocompatible oil in the inner core of the calibrated nanodroplets are displayed in Table 2, which shows a comparison between the sizes and PDI characterization of the different compositions obtained by microfluidic technique.

In particular, each Composition was prepared either by adding tricaprilin in the organic phase (Compositions 1B-D, 2B-D and 3B-D) or without (Compositions 1A, 2A and 3A) in order to observe any effect deriving from the presence of the oil in the inner core of the nanodroplets. Furthermore, three different oil amounts (2, 5 and 10 μL/mL) were tested to study the influence of the oil quantity on the nanodroplets properties.

The variation of the NDs sizes over time is expressed as % Evol, calculated following Equation 4, as described in the Description Section.

A value of % Evol close to 0 indicates a higher stability of the calibrated NDs suspension, whereby the nanodroplets in the suspension substantially maintain over time their initial mean dimension.

Results showed that the addition of a biocompatible oil endowed to an improved NDs stability (i.e. low % Evol) over time in all the tested compositions, in comparison with the compositions comprising only perfluorocarbon in the core.

In particular, it was observed that a similar stability was achieved by all the compositions comprising the biocompatible oil, notwithstanding the different molar ratios between the BFS and the PFC and the different amount of biocompatible oil.

Moreover also the biocompatible oil/fluorinated surfactant volume ratio was determined for Composition 1 (Table 2b).

As inferable from the results, the volume ratios between the biocompatible oil and the fluorinated compound was substantially high, with the biocompatible oil occupying up to 50% of the core volume.

TABLE 2b
Molar ratio and volume ratio between biocompatible
oil and fluorinated compound

| | | | | | | Volume |
|---|---|---|---|---|---|---|
| | | | | Molar | Molar | ratio |
| | | Oil | | ratio Nr | ratio Nr | % v/v |
| | | (μL/mL) | FRR | (BFS/PFC) | Oil/PFC | Oil/PFC |
| Composition 1 | B | 2 | 1:1 | 0.025 | 0.072 | 17% |
| | C | 5 | | | 0.179 | 33% |
| | D | 10 | | | 0.359 | 50% |

### Example 3

### Effect of the Biocompatible Fluorinated Surfactant Nature on the Stability of the Calibrated Dual-Phase Nanodroplets

In order to study the influence of biocompatible fluorinated surfactants on the NDs sizes and size distribution, different compositions comprising different BFS were tested, namely:
Compositions 1-6: comprising a nanodroplets suspension stabilized by the Dendri-TAC surfactant DiF₆DiTAC₇ and having a core comprising perfluoropentane as PFC and tricaprilin as biocompatible oil, and
Compositions 7-12: comprising a nanodroplets suspension stabilized by the F-TAC surfactant F₈TAC₁₈ and having a core comprising perfluoropentane as PFC and tricaprilin as biocompatible oil.

All the investigated compositions were prepared as described in Example 1, and the processing parameters were set as to obtain different molar ratios (from low to high) between BFS and PFC for each composition. For this purpose, three different FRR were tested: 3:1. 2:1 and 1:1. The TFR was 10 ml/min. For all the Compositions 1-12 the concentrations of perfluoropentane and tricaprilin in the organic phase were 10 μL/mL and 5 μL/mL, respectively. Two different concentrations of BFS in the aqueous phase were tested, namely 1 mg/ml (to obtain the lower molar ratios BFS/PFC) and 5 mg/ml (to obtain the higher molar ratios BFS/PFC).

A summary of all the Compositions obtained is listed in Table 3 (Compositions 1-6) and Table 4 (Compositions 7-12), wherein the quantities of biocompatible oil, the BPF/PFC molar ratios and the respective FRRs are displayed in the first left columns.

The obtained formulations were characterized using a Malvern Zetasizer Nano-ZS instrument (Malvern Instruments Ltd., 10 UK), measuring sizes (z-average) and polydispersity (PDI) over time after a storage of one week at 4° C.

Each measurement was performed at room temperature (i.e. 25° C.).

### Results

Tables 3 and 4 report the results obtained from the characterization of the microfluidically-prepared Compositions 1-12.

In order to observe any effect deriving from the presence of the oil in the inner core of the nanodroplets, each Composition was prepared either by adding tricaprilin in the organic phase (Compositions 1-6 C) or without (Compositions 1-6 A).

It was observed that using both DendriTAC and FTAC as BFS to stabilize the shell of the calibrated dual-phase nanodroplets allowed to obtain monodisperse NDs suspensions, characterized by homogenous nanodroplets in a range size of about 150-700 nm.

The variation of the NDs sizes over time is expressed as % Evol, calculated following Equation 4, as described in the Description Section.

A value of % Evol close to 0 indicates a higher stability of the calibrated NDs suspension, whereby the nanodroplets in the suspension substantially maintain over time its initial mean dimensions.

Results showed that the addition of a biocompatible oil endowed to an improved NDs stability (i.e. low % Evol) over time in all the tested compositions, in comparison with the compositions comprising only perfluorocarbon in the core (higher values of % Evol).

In particular, it was observed that a similar stability was achieved by all the compositions, stabilized by different biocompatible fluorinated surfactants and comprising the biocompatible oil, notwithstanding the different molar ratios between the BFS and the PFC and the different amount of biocompatible oil.

TABLE 3
Effects of the oil phase in the compositions of
calibrated nanodroplets stabilized by DendriTAC

| | | | Molar | | | | |
|---|---|---|---|---|---|---|---|
| | | | ratio | | | | |
| | | Oil | BFS/PFC | | Size | | % evol |
| | | μL/mL | nr | FRR | (nm) | PDI | (%) |
| Composition 4 | A | NO | 0.005 | 1-1 | 551 | 0.392 | 418 |
| | C | 5 | | | 304 | 0.075 | 1 |
| Composition 1 | A | NO | 0.025 | | 270 | 0.067 | 99 |
| | C | 5 | | | 680 | 0.258 | −17 |
| Composition 5 | A | NO | 0.010 | 2-1 | 280 | 0.161 | 67 |
| | C | 5 | | | 184 | 0.42 | −7 |
| Composition 2 | A | NO | 0.051 | | 198 | 0.108 | −85 |
| | C | 5 | | | 150 | 0.218 | 2 |
| Composition 6 | A | NO | 0.015 | 3-1 | 282 | 0.15 | 51 |
| | C | 5 | | | 159 | 0.287 | −5 |
| Composition 3 | A | NO | 0.076 | | 194 | 0.098 | 52 |
| | C | 5 | | | 144 | 0.257 | 1 |

TABLE 4
Effects of the oil phase in the compositions
of calibrated nanodroplets stabilized by FTAC

| | | | Molar | | | | |
|---|---|---|---|---|---|---|---|
| | | Oil | ratio | | | | % |
| | | μL/ | BFS/PFC | | Size | | evol |
| | | mL | nr | FRR | (nm) | PDI | (%) |
| Composition 7 | A | NO | 0.005 | 1-1 | 442 | 0.239 | 142 |
| | C | 5 | | | 397 | 0.214 | −11 |
| Composition 8 | A | NO | 0.024 | | 354 | 0.204 | 273 |
| | C | 5 | | | 365 | 0.087 | −3 |
| Composition 9 | A | NO | 0.010 | 2-1 | 280 | 0.168 | 169 |
| | C | 5 | | | 240 | 0.214 | −8 |
| Composition 10 | A | NO | 0.049 | | 215 | 0.169 | 426 |
| | C | 5 | | | 129 | 0.241 | 7 |
| Composition 11 | A | NO | 0.015 | 3-1 | 264 | 0.144 | very |
| | | | | | | | instable |
| | C | 5 | | | 238 | 0.177 | −10 |
| Composition 12 | A | NO | 0.073 | | 174 | 0.123 | 107 |
| | C | 5 | | | 117 | 0.29 | 7 |

### Example 4

### Effects of the Nature of the Biocompatible Oil on the Stability of the Calibrated Dual-Phase Nanodroplets

In order to study the influence of the nature of the biocompatible oil on the NDs stability, several compositions of dual-phase nanodroplets comprising different biocompatible oils were studied. For this purpose, two oils characterized by different lipophilic profiles were selected, namely tricaprilin (XLogP3-AA=8.9, computed by XLogP3 3.0 (PubChem release 2021 May 7)) and tributyrin (XLogP3-AA=2.4, computed by XLogP3 3.0 (PubChem release 2021 May 7)).

The following compositions were compared:
Compositions 7C, 9C and 11C: comprising a nanodroplets suspension stabilized by the F-TAC surfactant F₈TAC₁₈ and having a core comprising perfluoropentane as PFC and tricaprilin (5 μL/mL) as biocompatible oil;
Compositions 13, 14 and 15 comprising a nanodroplets suspension stabilized by F-TAC surfactant F₈TAC₁₈ and having a core comprising perfluoropentane as PFC and tributyrin (5 μL/mL) as biocompatible oil.

All the above compositions were prepared as described in Example 1, and the processing parameters were set as to obtain different molar ratios (from low to high) between BFS and PFC for each composition (i.e. 0.005, 0.010 and 0.015). Three different FRR were tested: 3:1. 2:1 and 1:1.

The samples were characterized using a Malvern Zetasizer Nano-ZS instruments in order to determine the NDs sizes (Z-Average) and polydispersity (PDI) over time after a storage of one week at 4° C.

Each measurement was performed at room temperature (i.e. 25° C.).

The ranges of the evaluated BFS/PFC molar ratios as function of the selected FRR are displayed in Table 5.

### Results

TABLE 5
Effect of the nature of the biocompatible
oil on the stability of the calibrated

| | | | Molar | |
|---|---|---|---|---|
| | | | ratio | |
| | | | BFS/PFC | % evol |
| | | FRR | nr | (%) |
| Tricaprilin | Composition 7C | 1:1 | 0.005 | −11 |
| (XLogP3- | Composition 9C | 2:1 | 0.010 | −8 |
| AA = 8.9) | Composition 11C | 3:1 | 0.015 | −10 |
| Tributyrin | Composition 13 | 1:1 | 0.005 | 69 |
| (XLogP3- | Composition 14 | 2:1 | 0.010 | 138 |
| AA = 2.4) | Composition 15 | 3:1 | 0.015 | 167 |

Table 5 reports the results obtained by the characterization of different calibrated dual-phase nanodroplets compositions, comprising different biocompatible oils.

In particular, the oil influence on the stability of the nanodroplets was investigated overtime, and the results were expressed as % Evol, calculated following Equation 3, as described in the Description Section.

The overall findings indicate that the presence of a biocompatible oil having a higher lipophilic profile, such as tricaprilin (XLogP3-AA=8.9) endowed to a significantly improved stability over time in comparison to a less lipophilic oil, such as tributyrin (XLogP3-AA=2.4).

In fact, Compositions 7C, 9C and 11C comprising tricaprilin were characterized by values of % Evol closer to 0 than those measured for Compositions 13, 14 and 15 comprising tributyrin, indicating a higher stability of the calibrated NDs suspensions, whereby the nanodroplets in the suspension substantially maintain over time its initial mean dimensions.

### Example 5

### Encapsulation of a Dye into the Calibrated Dual-Phase Nanodroplets

The ability of the disclosed calibrated dual-phase nanodroplets to encapsulate a dye into the inner core, was evaluated.

For this purpose, Compositions 4-6C were selected to be loaded with the fluorescent DIO dye. Composition 4-6C were prepared as described in Example 1, and the DIO dye was furtherly added at a concentration of 0.0625 mg/mL into the organic phase before its injection into the microfluidic cartridge. The processing parameters were set as to obtain different molar ratios (from low to high) between BFS and PFC for each composition.

Three different FRR were tested: 3:1. 2:1 and 1:1.

At the end of the microfluidic preparation, the nanodroplets compositions were centrifuged at 5000 g for 12 minutes in order to separate the pellet, containing the DIO-encapsulated nanodroplets, from the nanodroplets-free supernatant containing the non-encapsulated DIO.

Both the pellet and the supernatant were then analysed aiming at assessing the amount of DIO encapsulated into the nanodroplets using a fluorometer Cytation 5 (Cell Imaging Multi-Mode Reader)

### Results

Table 6 showed the overall results obtained by the determination of the amount of the DIO encapsulated in the calibrated dual-phase nanodroplets.

All the investigated compositions were able to incorporate DIO into the pellet, confirming the encapsulation ability of the disclosed calibrated dual-phase nanodroplets, forming the pellet.

TABLE 6
Amount of DIO encapsulated into the
calibrated dual-phase nanodroplets

| | | Molar ratio | Amount of |
|---|---|---|---|
| | | BFS/PFC | encapsulated dye |
| | FRR | nr | % |
| Composition 4C | 1:1 | 0.005 | 48 |
| Composition 5C | 2:1 | 0.010 | 30 |
| Composition 6C | 3:1 | 0.015 | 28 |

Moreover, the amount of DIO dye encapsulated into the nanodroplets (pellet) was assessed to be between 28 and 48% of the quantity of DIO added during the nanodroplets preparation. This amount is a significant improvement if compared with the work of Al Rifai et al 2020, indicating that using the disclosed microfluidic method is possible to reduce the amount of dye been lost during the nanodroplets preparation, that usually is caused by the centrifugation procedures.

### Example 6

### Determination of Acoustic Droplet Vaporization (ADV)

The expression “Acoustic Droplet Vaporization (ADV) threshold” indicates the minimal acoustic pressure that is necessary to obtain the nanodroplets conversion into echogenic microbubbles.

The Acoustic Droplet Vaporization (ADV) threshold of the calibrated dual-phase NDs prepared according to the previous examples can be determined according to conventional methodologies using B-mode imaging methods. For instance, the suspension of NDs can be vaporized while passing through the focal zone of a transducer and the acoustic pressure is increased of about 0.2 MPa each 5 s until the NDs vaporization is observed.

Nanodroplets activation was performed by focused ultrasound waves on five aligned focal points allowing the activation only within the region of interest where the acoustics pressure was highest. Pulses were emitted in burst mode at a frequency of 6 MHz, 20 cycles per pulse and at a pulse-repetition frequency (PRF) of 1 Hz.

The acoustic pressure was increased every 5 s until the observation of the NDs vaporization.

For the ADV determination, two different compositions were tested: the Composition 1A, not comprising biocompatible oil, and Composition 1D, comprising tricaprilin. For all experiments, 0.4 mL of each NDs suspensions were diluted in 40 ml of water at 37° C.

### Results

Table 7 reports the overall results obtained from the determination of the ADV thresholds. Each value is the average of three successive determinations.

TABLE 7
Acoustic Droplet Vaporization (ADV) threshold as
a function of the presence of biocompatible oil

| Composition | Oil | MPa |
|---|---|---|
| Composition 1A | NO | 7.93 |
| Composition 1D | 10 μL/mL | 8.14 |

From the comparison, it emerges that the two tested compositions had similar ADV threshold values, confirming their similar acoustic response.

### REFERENCES

Astafyeva et al, J. Mater. Chem. B, 3, 2015, 2892-2907 WO2016185425
Al Rifai et al, J. Mater. Chem. B, 8, 2020, 1640-1648
Melich et al., International Journal of Pharmaceutics, 587, 2020, 119651
Cheng et al, J. Chem. Inf. Model. 2007, 47, 2140-2148.
Helfield et al., Ultrasound in Medicine & Biology, 46, 10, 2020, 2861-2870

1. A nanodroplet comprising an outer layer and an inner core, said outer layer comprising a biocompatible fluorinated surfactant and said inner core comprising a fluorinated compound and a triglyceride having a logP value higher than 5, wherein said biocompatible fluorinated surfactant is selected from:
(A) an amphiphilic dendrimer of generation n comprising:
a hydrophobic central core of valence 2 or 3;
generation chains attached to the central core and branching around the core; and
a hydrophilic terminal group at the end of each generation chain;
wherein
n is an integer from 0 to 12;
the hydrophilic terminal group comprises:
a mono-, oligo- or polysaccharide residue,
a cyclodextrin residue,
a peptide residue,
a tris(hydroxymethyl)aminomethane (Tris), or
a 2-amino-2-methylpropane-1,3-diol;
the hydrophobic central core being a group of formula (Ia) or (Ib): 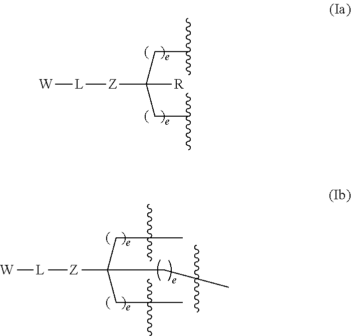
wherein:
W is R_{F} or a group selected from W₀, W₁, W₂ or W₃: 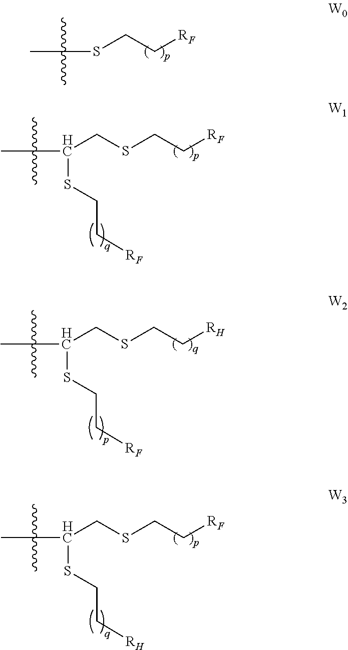
R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—,
Z is C(═O)NH or NHC(═O),
R is a C₁-C₆ alkyl group, and
e is at each occurrence independently selected from 0, 1, 2, 3 or 4;
(B) an amphiphilic linear oligomer of formula II 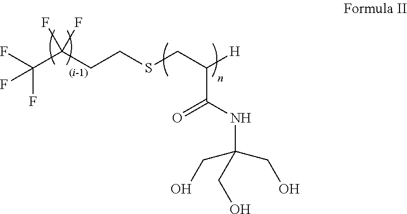
wherein:
n is the number of repeating Tris units (n-DPn is the average degree of polymerization), wherein the term Tris indicates the tris(hydroxymethyl)aminomethane unit, and
i is the number of carbon atoms in the fluoroalkyl chain
or a mixture thereof.

2. The nanodroplet according to claim 1, wherein said fluorinated compound is a perfluorocarbon.

3. The nanodroplet according to claim 1, wherein said triglyceride has a logP value higher than 7 up to 25.

4. The nanodroplet according to claim 1, wherein said triglyceride is tricaprilin, trilaurin, triolein, trilinolein or a mixture thereof.

5. An aqueous suspension comprising a plurality of nanodroplets according to claim 1, wherein said nanodroplets have a z-average diameter comprised between 100 nm and 1000 nm and a polydispersity lower than 0.25.

6. A method for the preparation of an aqueous suspension comprising a plurality of nanodroplets, said nanodroplets comprising an outer layer and an inner core, said outer layer comprising a biocompatible fluorinated surfactant as defined in claim 1, and said inner core comprising a fluorinated compound and a biocompatible oil having a logP value higher than 5, said method comprising the steps of:
a) preparing an aqueous phase;
b) preparing an organic phase,
wherein
i) said aqueous phase comprises a biocompatible fluorinated surfactant as defined in claim 1 or a mixture thereof and the organic phase comprises a fluorinated compound and a biocompatible oil having a logP value higher than 5 or
ii) said organic phase comprises a biocompatible fluorinated surfactant as defined in claim 1 or a mixture thereof, a fluorinated compound and a biocompatible oil having a logP value higher than 5;
c) injecting said aqueous phase into a first inlet and said organic phase into a second inlet of a microfluidic cartridge, thereby mixing said aqueous phase and said organic phase in a mixing device of the microfluidic cartridge, wherein the operating pressure into said microfluidic cartridge is lower than 7000 kPa, to obtain said aqueous suspension; and
d) collecting said aqueous suspension from the exit channel of the microfluidic cartridge.

7. The method according to claim 6, wherein said aqueous phase comprises said biocompatible fluorinated surfactant and said organic phase comprises a fluorinated compound and a biocompatible oil having a logP value higher than 5.

8. The method according to claim 6, wherein said fluorinated compound is a perfluorocarbon.

9. The method according to claim 6, wherein said biocompatible oil has a logP higher than higher than 7 up to 25.

10. The method according to claim 6, wherein said biocompatible oil is a triglyceride.

11. The method according to claim 10, wherein said triglyceride is selected from tricaprilin, trilaurin, triolein, trilinolein or a mixture thereof.

12. The method according to claim 6, wherein the ratio between the volume of said aqueous phase and the volume of said organic phase is comprised between 1:1 to 5:1.

13. The method according to claim 6, further comprising additional step e) wherein said collected aqueous suspension is diluted.

14. The method according to claim 6, wherein said aqueous suspension comprises a plurality of nanodroplets having a z-average diameter comprised between 100 nm and 1000 nm and a polydispersity lower than 0.25.

15. A method of diagnostic and/or therapeutic treatment comprising administering the aqueous suspension according claim 5 to a patient.

16. The nanodroplet according to claim 1, wherein said triglyceride has a logP value higher than 9 up to 25.

17. The method according to claim 6, wherein said biocompatible oil has a logP higher than higher than 9 up to 25.

18. The method according to claim 6, further comprising additional step e) wherein said collected aqueous suspension is diluted with water.
